(19) United States
(12) Patent Application Publication
(10) Pub. No.: US 2024/0408368 A1
(43) Pub. Date: Dec. 12, 2024

(54) APPLICATOR FOR MEDICAMENT PATCH

(71) Applicant: VAXESS TECHNOLOGIES, INC., Cambridge, MA (US)

(72) Inventors: Shehryar Siddiqui, Hopkinton, MA (US); Elizabeth Whitney Johansen, Hanover, MA (US); Nickolas W. Hartman, Houson, TX (US); Matthew Dirckx, Medford, MA (US)

(73) Assignee: VAXESS TECHNOLOGIES, INC., Cambridge, MA (US)

(21) Appl. No.: 18/806,128

(22) Filed: Aug. 15, 2024

Related U.S. Application Data
(63) Continuation of application No. PCT/US2023/026036, filed on Jun. 23, 2023.
(60) Provisional application No. 63/355,301, filed on Jun. 24, 2022.

(51) Int. Cl. A61M 37/00; A61K 9/70

(52) U.S. Cl. CPC A61M 37/0015; A61K 9/703; A61M 2037/0023

(57) ABSTRACT

Patch applicator devices are provided. The devices can include a reusable or single-use system for applying a push-through or breakaway patch to a patient's skin. The devices can improve application and effectiveness of microneedle based patches.

This application is a continuation application of International Application No. PCT/US2023/026036, filed on Jun. 23, 2023, which claims the benefit of priority to U.S. Provisional Application No. 63/355,301, filed on Jun. 24, 2022, the entire contents of each of which are expressly incorporated herein by reference.

This invention was made with government support under R44AI142948and SB1AI164584 awarded by the National Institutes of Health. The government has certain rights in the invention.

The present disclosure relates generally to applicator devices and systems for medicament patches. The devices and systems can be used to reliably apply patches to a patient's skin.

There are currently numerous devices available or being developed for delivery of medications (e.g., an influenza vaccine and/or a coronavirus vaccine, e.g., an mRNA-based vaccine) to patients through the skin. The simplest of these devices can include systems that release or apply substances to the skin for absorption. More complicated systems can facilitate delivery by various techniques such as use of small needles to facilitate delivery deeper within the skin, or other systems, such as iontophoresis to encourage movement of materials into the skin.

Recently, patch-types devices have been developed that allow placement of biodegradable needle-like devices within the skin. These devices may carry substances to be delivered and degrade slowly over time, thereby exposing the body to the delivered substance over a desired time period, potentially along with other agents that protect the substance to be delivered or provide other therapeutic benefits (e.g., control release rate, improve biologic function). Such devices can be used for delivery of vaccines, small molecule drugs, biologics, combination products, or other therapeutic or prophylactic substances.

The effectiveness of such biodegradable needle-like patches can be improved by ensuring that the patches are applied reliably with sufficient force to deposit the biodegradable needles at a desired depth within the skin. However, although such patches may be effectively applied by simple manual application by a patient or health care provider, it would be beneficial to provide improved systems to reliably apply the biodegradable patches with little or minimal training and with a high level of repeatability.

Accordingly, the present disclosure provides improved devices for application of medical patches, including biodegradable needle patches.

### SUMMARY

The present disclosure relates to applicator devices for applying patch-type devices to a patient's skin. The devices can allow reliable application of a patch, including even and reliable application such that a sufficient force and/or depth of skin penetration is achieved to ensure that needle-like portions of the patch are positioned at a desired depth within or beneath a portion of the skin. The applicator can be configured to provide a predetermined force to quickly and reliably apply the patch to a desired location, thereby helping to improve delivery of active agents (e.g., drugs, vaccines, biologics, or other materials) using the selected patch

The applicator can include an outer body portion having a substantially cylindrical shape and a hollow interior; a piston slidably connected with the hollow interior of the outer body portion; a compressible member positioned within the outer body and configured to apply downward pressure to the piston; and an actuator positioned below the piston and being slidably engaged with the hollow interior and extending from a bottom portion of the outer body such that upward pressure on the actuator pushes the piston upward and compresses the compressible member. The piston and hollow interior of the outer body portion are slidably connected via a protrusion and a cam path, and the cam path forms a continuous loop such that upward pressure on the piston causes the piston to compress the compressible member moving the piston upward into the hollow interior of the outer body portion until the protrusion reaches a top portion of the cam path and engages a downward directed portion of the cam path, thereby releasing the piston into a downward portion of the continuous loop to release the piston and force the piston downward. A medicament patch is held in a ring holder at a bottom portion of the actuator and is pushed downward through the ring holder and onto an object held in contact with a bottom surface of the ring holder.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a perspective view of a medicament patch applied to a patient's arm.

FIGS. 2A and 2B are side and bottom perspective views respectively of an exemplary applicator system for use with a medicament patch like that illustrated in FIG. 1.

FIG. 3 is a perspective view of an exemplary applicator showing internal components.

FIG. 4A is a side view of the embodiment of FIG. 3 with the compressible member and piston in a down position.

FIGS. 4B and 4C are additional views of the exemplary applicator of FIG. 3 and FIG. 4A in a partially compressed position before deployment.

FIG. 4D is an additional view of the exemplary applicator of FIGS. 3, 4A, 4B, and 4C after release of the piston to deploy the device and apply the patch.

FIGS. 5A-5C are perspective, top, and bottom views of a ring holder for a medicament patch according to exemplary embodiments.

FIGS. 6A and 6B are exemplary patch configurations for use with disclosed applicator and patch systems.

FIG. 7 is a side view of one embodiment of a ring hold for a medicament patch attached to an actuator of a patch applicator.

FIG. 8 is a perspective view of a retainer mechanism for a medicament patch to be used with a patch applicator.

FIG. 9A is a perspective view of another retainer mechanism for a medicament patch to be used with a patch applicator.

FIGS. 9B and 9C are side cut-away and cut-away exploded views of the retainer mechanism of FIG. 9A.

FIGS. 10A and 10B are side views illustrating the movement path and connection mechanism of the piston of an exemplary patch applicator device.

FIGS. 11A and 11B are perspective and side views illustrating the connection and movement of the actuator within an exemplary patch applicator device.

FIG. 12 is a perspective view of the actuator illustrated in FIGS. 11A and 11B.

FIG. 13A is a perspective view of a piston of an exemplary patch applicator device.

FIG. 13B is a top view of a piston of an exemplary patch applicator device.

FIGS. 14A-14C illustrate interaction between an actuator, outer body portion, and piston of the applicator, according to various embodiments.

FIG. 15 is alternative break-away mechanism for holding a medicament patch in a patch applicator.

FIGS. 16A-C are alternative exemplary embodiments for a backing portion of a medicament patch.

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purposes.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

As noted above, various patch devices are available for delivery of medications or other substances into or through the skin. As used herein, “medicament” or “medicament patch” will be understood to refer to any substance or patch carrying a substance to provide a biologic effect to a patient. “Medicaments” will be understood to refer to any pharmaceutical, small molecule drug, vaccine (including any vaccine such as an mRNA vaccine, protein, glycoprotein, live viral, live attenuated viral, inactivated viral, recombinant, or other vaccine) peptide, biologic, antibody, vitamin, mineral, hormone, or other materials that can be delivered into or through the skin.

Medicament patches can include micro-needle based devices, which are described in more detail below. Such microneedle devices can include one or more (and preferably a group or an array) of microneedles. The microneedles are located on a skin-facing surface of a flexible or semi-rigid patch, and by application of the patch to a patient's skin, the needles can penetrate the skin to a desired depth. In some cases, the microneedles may include a biodegradable component that is deposited at a desired distance into the skin and may degrade at a desired rate to release or present the medicament to a patient, thereby eliciting a desired response, such as an immunologic response to a vaccine. More details regarding exemplary patches, including micro-needle patches are described further below.

In order to improve the efficacy of any medicament patch, it is desirable to ensure that the patch is applied appropriately, including even application to ensure that as many microneedles as possible are positioned at an appropriate depth. Furthermore, it is desirable to ensure that the patch is applied such that the microneedles are pushed into the skin at a desired depth without inadvertent shearing of microneedles above the skin. Proper application of the patch can improve overall efficacy.

In order to improve consistency and effectiveness of patch application, an automated applicator device may be desirable. Accordingly, the present disclosure provides embodiments of an applicator device having one or more features or advantages over existing devices or simple manual application. The disclosed devices may be configured for repeated or single use, and the devices may be pre-loaded with a patch (i.e., as a kit or patch product including applicator and patch). Alternatively, the applicator may be separate from a patch, and a patch may be selected and loaded to the device (e.g., based on desired medicaments, patient characteristics, or need for additional applications for more than one patient or for a patient that needs more than one patch).

The applicator can improve patch application by one or more of (1) properly holding and/or stretching/pre-tensioning skin to receive the patch, (2) applying a reliable degree of force and/or depth of force against the skin to ensure proper microneedle placement, and/or (3) controlling distribution of application force across the patch. The structure and function of the applicator in its various embodiments is described in more detail below.

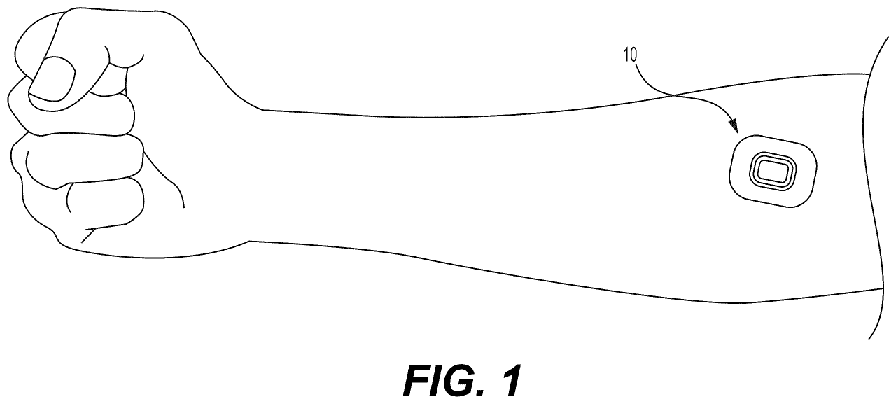

FIG. 1 is a perspective view of a medicament patch 10 applied to a patient's arm. As shown, the patch 10 is substantially square, or square with rounded corners, but other shapes and configurations are contemplated and described below. The patch will generally have an adhesive to secure the patch to a patient's skin. At least the backing layer will be semi-flexible and capable of being deployed using the applicator(s) described herein.

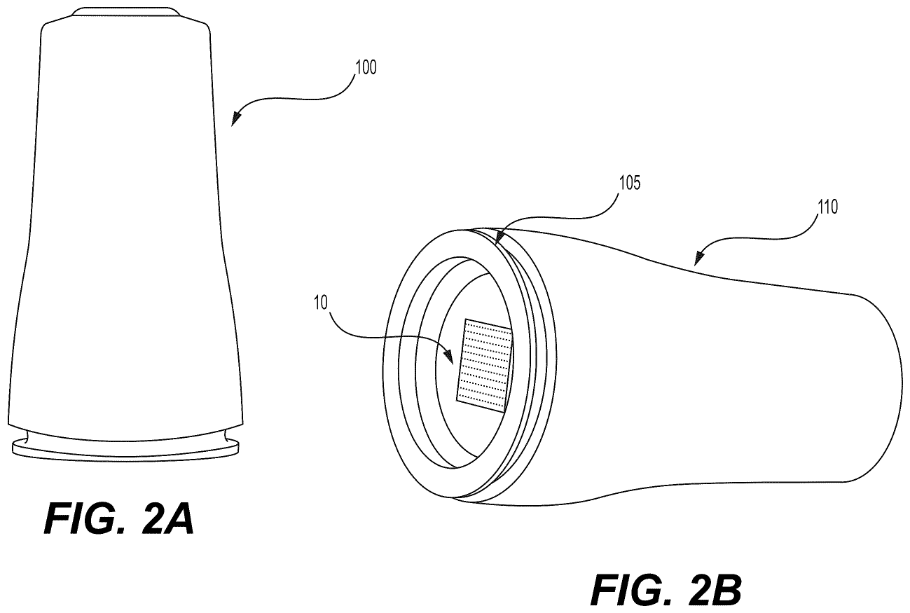

FIGS. 2A and 2B are side and bottom perspective views respectively of an exemplary applicator 100 for use with a medicament patch 10 like that illustrated in FIG. 1. As shown, the applicator 100 has a main upper body portion 110 and lower patient contacting portion 105 (e.g., a bottom surface 120 of actuator 122 or a ring holder 50, described below). The upper main body portion 110 can be held by a user, and the patient contacting portion 105 can be pressed against a patient's skin. As the device is pressed against the patient's skin a potential energy is generated until sufficient pressure is applied, at which point an internal piston system is pushed downward onto the top of the patch 10 to deploy the patch correctly onto the patient's skin. Specific details of the applicator internal components and their function will be described with the following figures.

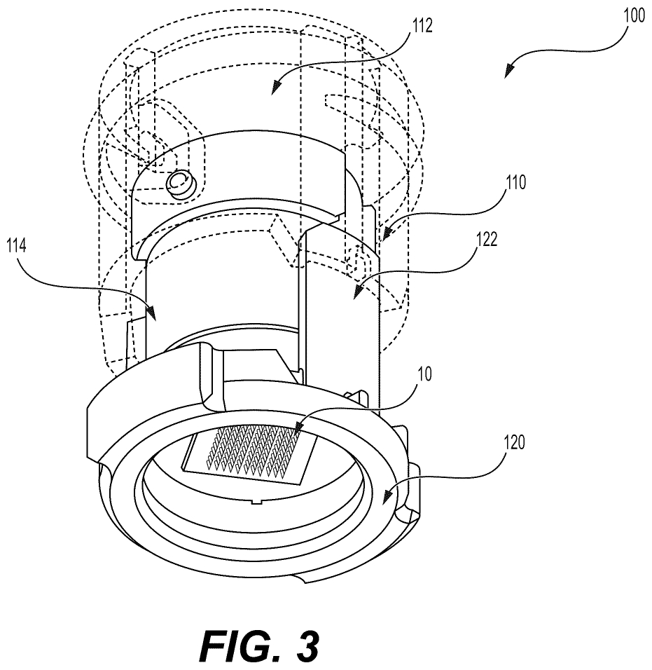

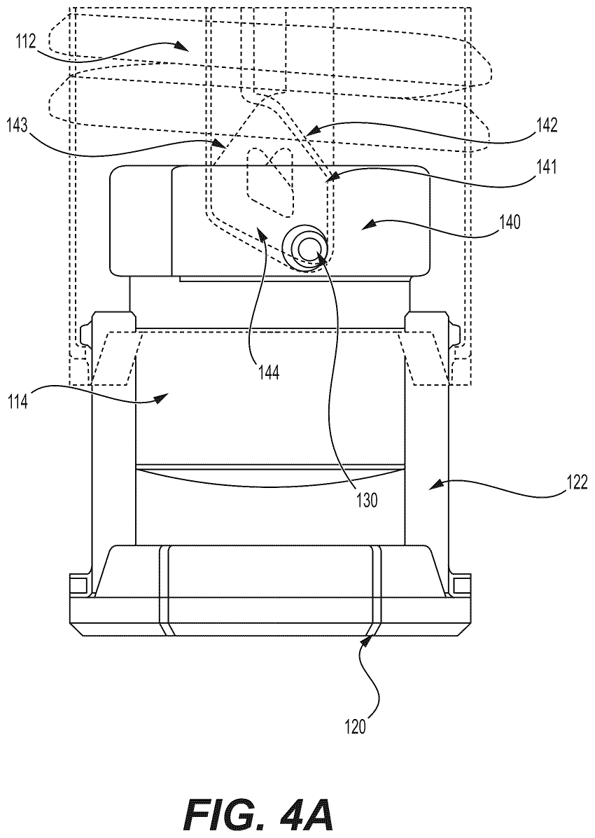

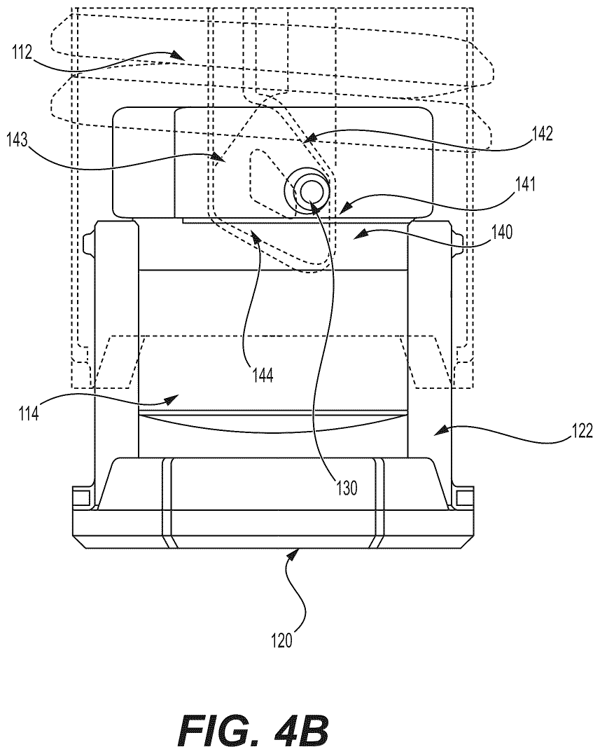

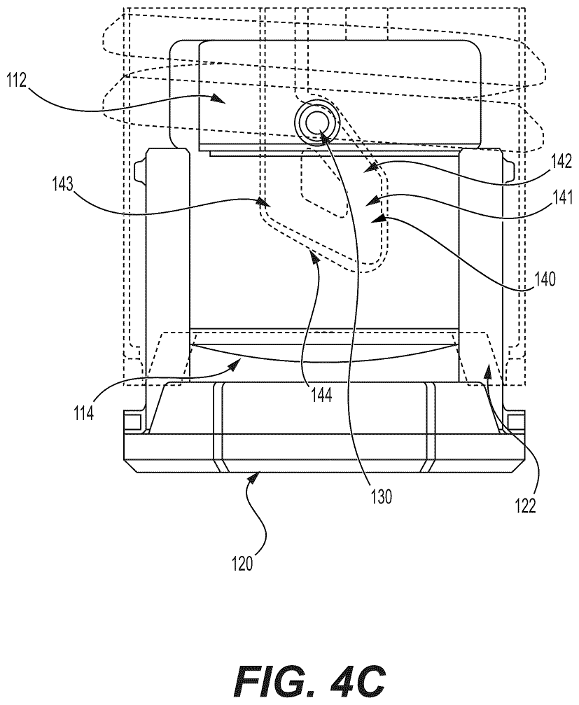

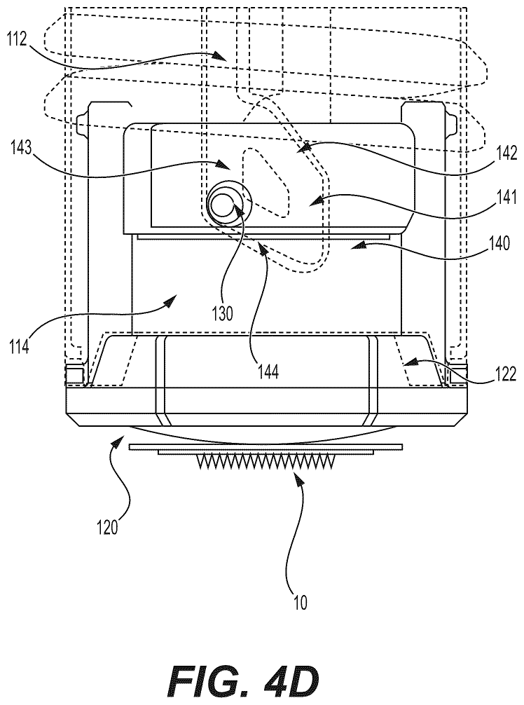

FIGS. 3 and 4A-4D illustrate detailed assembled components and operation of the applicator 100, according to various embodiments. Specifically, FIG. 3 is a perspective view of an exemplary applicator 100 showing internal components. FIG. 3 illustrates the patch applicator 100 including a patch 10, which is in the form of a generally flexible sheet.

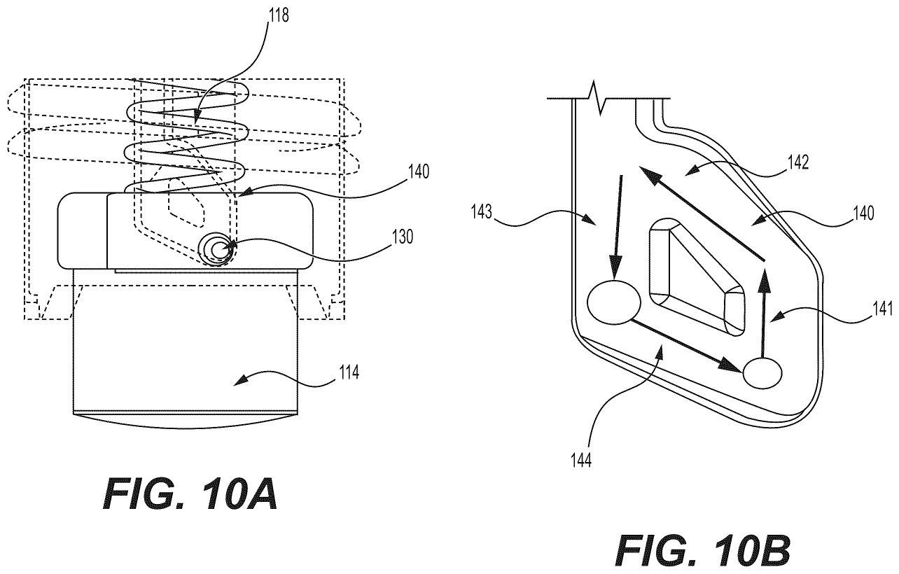

The applicator 100 includes an outer body portion 110 having a substantially cylindrical shape and a hollow interior 112. A piston 114 is slidably connected with the hollow interior 112 of the outer body portion. A compressible member 118 (illustrated in FIG. 10A) is positioned within the outer body 110 and is configured to apply downward pressure to the piston 114. An actuator 122 is positioned below the piston 114 and is slidably engaged with the hollow interior 112. The actuator extends from the outer body 100 such that upward pressure on a bottom area 120 of the actuator 122 pushes the piston 114 upward and compresses the compressible member 118;

The compressible member 118 can be any suitable spring or other compressible structure. For example, the spring may be a typical spring, compression spring, wave spring, dome spring, or leaf spring. Alternatively, a compressible member such as a balloon, compressible bladder, or a similar structure may be used.

As explained with reference to FIGS. 4A-4D, the piston 114 and hollow interior 112 of the outer body portion are slidably connected with a protrusion 130 and a cam path 140, and the cam path forms a continuous loop such that upward pressure on the piston causes the piston 114 to compress the compressible member 118 moving the piston upward into the hollow interior of the outer body portion until the protrusion reaches a top portion of the cam path and engages a downward portion of the cam path, thereby releasing the piston into a downward portion of the continuous loop to release the piston and force the piston downward. Further, the medicament patch 10 is held in a ring holder near a bottom portion of the actuator 122 and is pushed downward through the ring holder and onto an object held in contact with a bottom surface of the ring holder when the applicator is activated.

Turning now to FIGS. 4A-4D to explain operation of the applicator components, FIG. 4A is a side view of the embodiment of FIG. 3 with the compressible member 118 and piston 114 in a down position. FIGS. 4B and 4C are additional views of the exemplary applicator of FIG. 4A in a partially compressed position before deployment. FIG. 4D is an additional view of the exemplary applicator of FIGS. 3, 4A, 4B, and 4C after release of the piston to deploy the device and apply the patch.

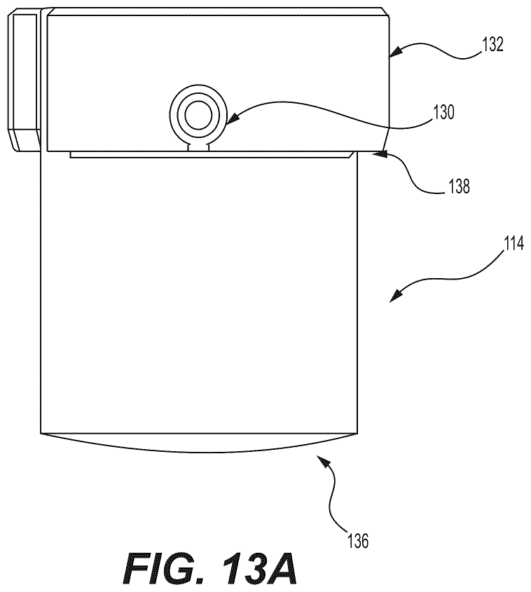

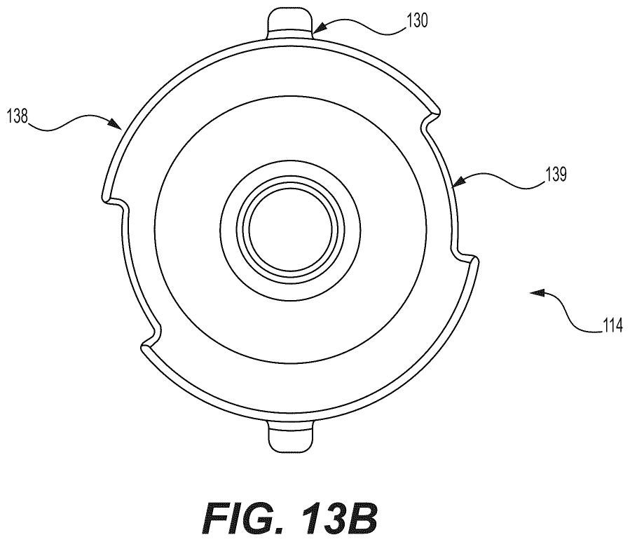

As shown, the piston 114 has a protrusion 130 on at least one side that engages a cam path 140 on an internal surface of the outer body 110. The protrusion 130 is also illustrated in FIG. 13A, which provides additional separate details on piston embodiments. As shown, the protrusion 130 is located near the top portion 132 of the piston 114, but the positioning of the protrusion may be modified. In some cases, and generally, the piston 114 will contain more than one protrusion, and each protrusion will engage a separate cam path 140 on the internal surface of the outer body 110. For example, in one embodiment, the piston will have two protrusions, like that shown in FIG. 13A, and the two protrusions will be located on opposite sides of the piston. As such, each protrusion will engage a separate cam path 140 on opposite sides of the internal surface of the outer body 110. It is contemplated that more than two protrusions and cam paths could be used (e.g., three or protrusions spaces evenly around the piston with an equal number of cam paths). However, generally two protrusions will be used, as such an embodiment will adequately control movement of the piston 114.

Turning now to the specific configuration of the cam path 140. Movement of the protrusion 114 in the cam path 140 is illustrated in FIGS. 4A-4D. As shown, the cam path forms a continuous loop. For example, as shown, the loop includes an first upward section 141, a second upward sloped section 142, a third downward directed section 143, and a fourth downward sloped section 144. The sections 141-144 are ordered based on the order of movement during normal operation of the applicator 100. The movement path of the protrusion 130 and cam path 140 are illustrated in larger views in FIGS. 10A and 10B.

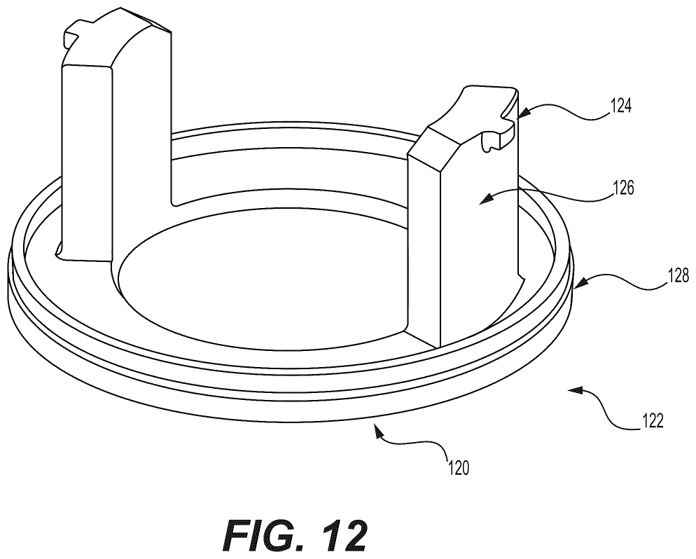

FIG. 4A illustrates the applicator 100 before use with the actuator 122 in a down position. As shown, the protrusion 130 is at a lowest point in the cam path 140, and accordingly, the piston 122 is in a low position. As upward pressure is placed on the actuator 122, i.e., by pushing the device against a patient's skin, the actuator moves upward and reaches a point where extensions 126 of the actuator (FIG. 12) engage a flange or widened region 138 of the piston 114, as illustrated in FIG. 13A. With continued pressure on the actuator 122, the piston 114 begins to move upward along the first section of the cam path 141, thereby compressing the compressible member 118. With continued upward pressure, the protrusion 130 of the piston moves along the second section 142 of the cam path 140, and accordingly, the piston 114 continues to move further upward to compress the compressible member.

Eventually, as shown in FIG. 4C, the protrusion nears the third downward directed section 143 of the cam path. Upon reaching the third downward directed portion 143 of the cam path, the protrusion 130 is able to move downward because the cam path is directed downward, and as the protrusion 140 reaches the third downward section 143 of the cam path 140, the actuator extensions 126 disengage from the flange or widened region 138 of the piston. The specific structure and function of the piston 114 and actuator 122 are explained in more detail below. In some cases, the device 100 can be reset or configured for additional use. In some cases, the holder is removed and the actuator is pulled to reposition and reset the device. In some cases, the devices is configured to allow only a single use.

As shown in FIG. 4D, as the piston moves downward, the piston 114 engages the patch 10, putting quick and sufficient pressure on the patch to push the patch 10 through a ring holder of the applicator and onto a patient's skin. The actual force applied can vary based on the specific patch configuration and target area on a patient. Generally, the piston will apply between about 90 N to 130 N to the patch against a target area of a patient's skin.

Although the cam path 140 is illustrated with four sections that form a loop in a shape that closely resembles a parallelogram, it is contemplated that the cam path 140 can have other shapes. For example, any shape forming a loop to allow repeated movement of the protrusion through the cam path 140 (i.e., to allow repeated use of the applicator 100) may be used. For example, suitable shapes may be more rounded and closer to an oval shape, but generally the cam path should have a downward directed section similar to that of fourth section 143 as it is the rapid motion of the protrusion along fourth section 143 that allows quick downward movement of the piston 114 to push the patch downward out of the applicator.

Generally, to allow the patch to be oriented correctly with respect to a target area of a patient's skin, and to provide a configuration wherein the piston 114 can push the patch off the applicator 100, the patch 10 is held in a ring type holder. Specifically, the patch is held by a holder that has a generally open region and a rigid rim or periphery. The patch is secured at one or more points along the patch's edge to the inner rim of the ring holder with the patch spanning the open region. The piston passes through the open region when activated to push the patch downward.

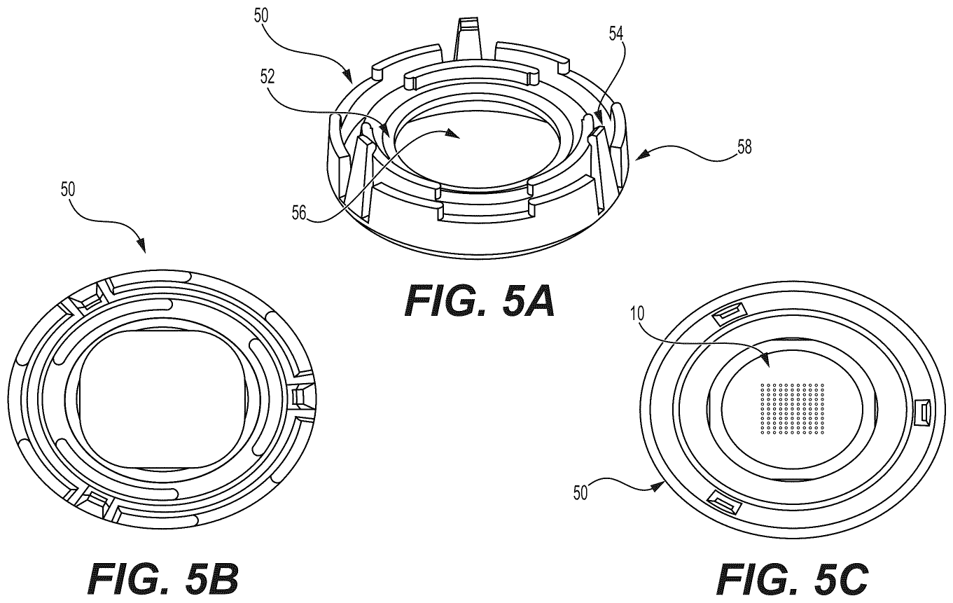

Specific embodiments of the ring holder are discussed below. For example, FIGS. 5A-5C are perspective, top, and bottom views of a ring holder 50 for a medicament patch 10 according to exemplary embodiments. The holder has a rigid peripheral rim 58 and central opening 56. The patch is secured over the opening 56 and to an inner part of the rim 58, as shown in FIGS. 5B and 5C. In some cases, the patch 10 is held at one or more points along its edges with a retainer ring 52.

As shown, the holder 50 is circular, but the use of the term ring is not meant to imply that the holder must be circular. The holder may have other shapes such as a square, oval, triangle, or other shape depending on such factors as the patch shape and configuration.

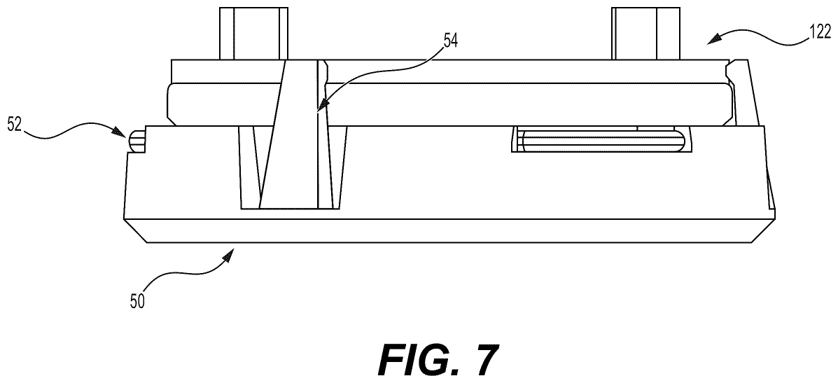

The ring holder 50 may be secured to a bottom portion 120 of the actuator 122 using a number of connection mechanisms. For example, the holder 50 and actuator 122 can be attached via extensions 54 with clips, as illustrated in FIG. 7. Other connection mechanisms such as a threaded, press-fit, friction fit, or adhesive connections may be used.

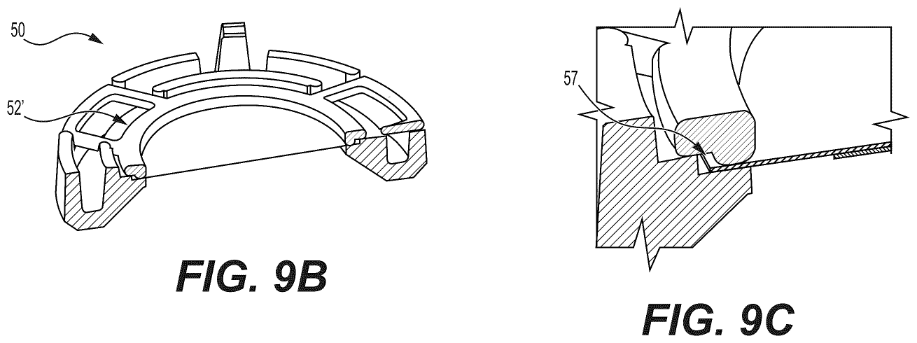

Variations on the ring holder configuration and how the patch is held are contemplated. For example, FIGS. 5A-5C illustrated a round retention ring 52, but the retention ring may have other shapes, including a ring 52′ (FIGS. 9A-9C) having extensions 53 to assist with removal and placement on the holder. Furthermore, the retention ring 52′ may have form a sandwich connection 57 (FIG. 9c) with a flange and groove or similar structure to help stabilize the connection between the retention ring 52′ and patch.

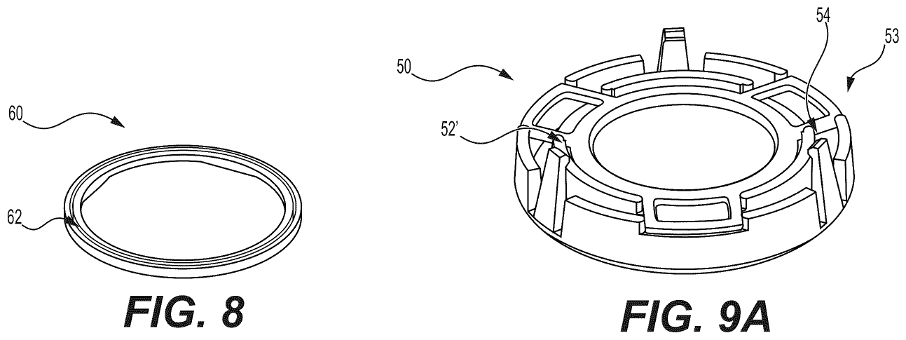

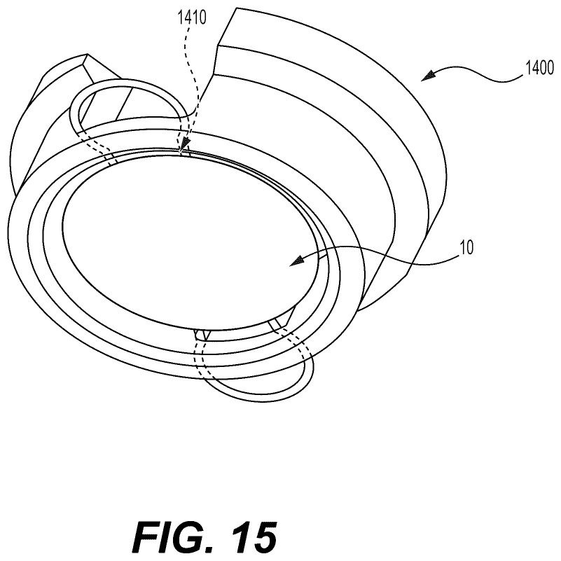

Furthermore, although the patch can be held in a holder attachable to a bottom portion of the actuator 122, other configurations are contemplated. For example, a small ring holder 60 with retainer ring 62 as shown in FIG. 8, may be used, and such a holder may be placed on a top portion of the actuator, thereby avoiding a larger ring holder attached to the bottom of the actuator. In addition, rather than using a retention ring, the patch 10 may be held with breakaway connectors. For example, FIG. 15 is alternative break-away mechanism 1400 for holding a medicament patch in a patch applicator. In the break-away mechanism, thin or relatively weak connectors 1410 hold the patch to the applicator, and the connectors 1410 are fractured or torn by pressure of the piston.

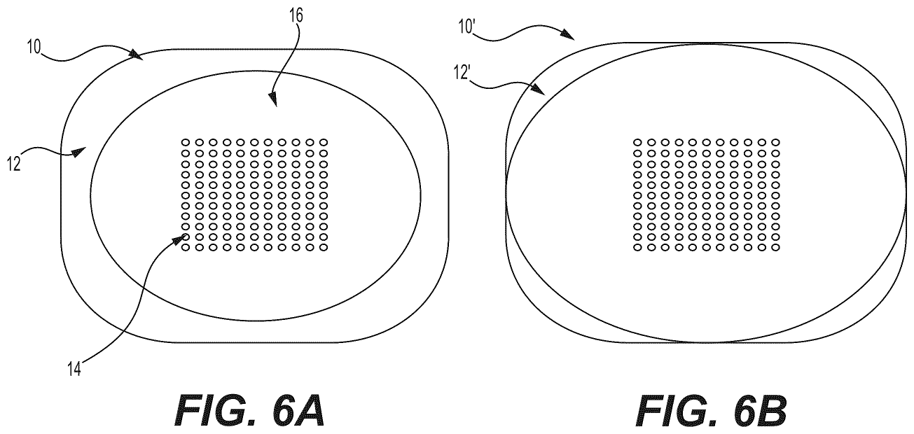

Suitable patches should be designed to be released from the actuator by application of pressure from the piston 114. FIGS. 6A and 6B are exemplary patch configurations for use with disclosed applicator and patch systems. As shown, the patch 10, 10′ can include a backing layer 12, 12′, and adhesive region 16, and an area containing medicament 14. The adhesive region 16 may extend into the area containing medicament 14 (e.g., a microneedle array) so long as the adhesive is not placed in a manner that adversely affected the microneedles or their detachment into the skin.

The backing layer can extend from the periphery of the adhesive region 16 along the entire periphery, as shown in FIG. 6A, or from a portion of the adhesive, e.g., at corners, as shown in the FIG. 6B. As discussed above, at least a portion of the patch, e.g., the backing layer, may have sufficient flexibility to allow the patch to be pushed through the ring holder of the applicator device 100. To provide sufficient flexibility, the backing layer can be formed of materials having mechanical properties and or dimensions that provide a desired degree of flexibility. For example, suitable materials for the backing layer can be polyesters (e.g., polyethylene terephthalate or polyethylene terephthalate glycol between about 0.002′ or 0.005″ thick), paper, aluminum or other flexible materials.

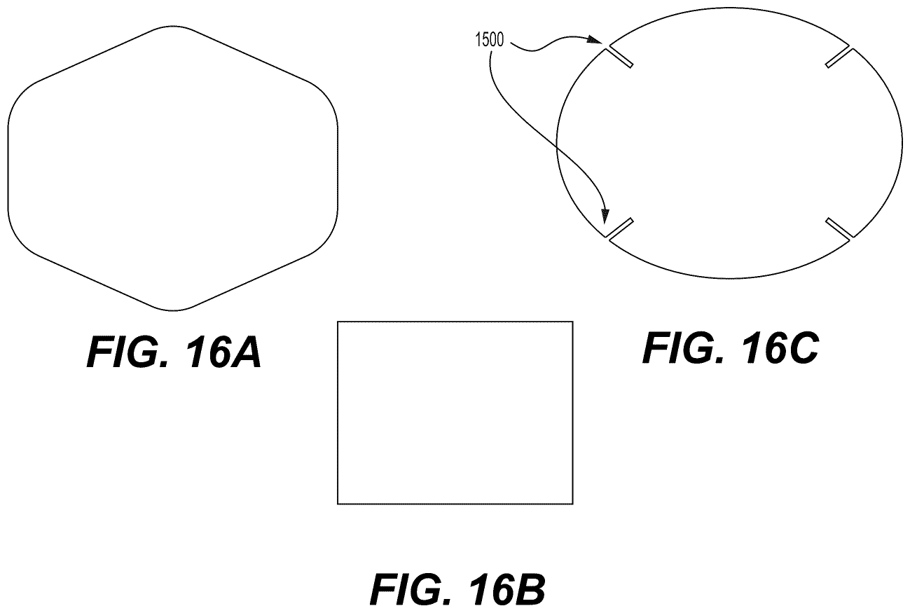

Furthermore, the patch 10, including the entire patch or backing layer, can have a variety of shapes. For example, FIGS. 16A-C are alternative exemplary embodiments for a backing portion of a medicament patch. The patches include polygon (e.g., hexagon (FIG. 16A) or octagon), square (FIG. 16B), or circular (FIG. 16C). Further, other shapes are contemplated such as triangles, ovoid, square with rounded edges (scround or squircle) as shown in FIGS. 6A or 6B. In addition, the backing layers can be modified to allow increased flexibility in certain areas. For example, cuts or indentations 1500 can be provided at certain areas along the backing layer periphery, as shown in FIG. 16C.

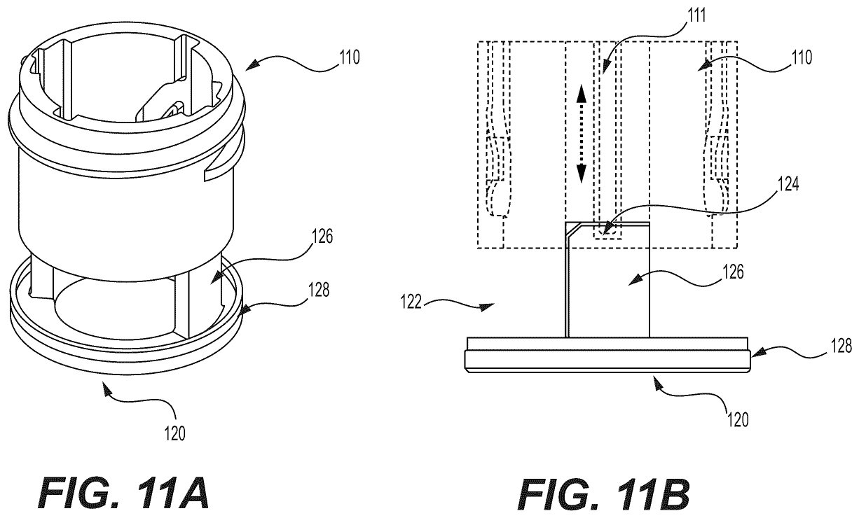

FIGS. 11A, 11B, 12, 13, and 14 provide more detail relating to the structure and interaction of the actuator 122 and piston 114, according to various embodiments. FIGS. 11A and 11B are perspective and side views illustrating the connection and movement of the actuator 122 within an exemplary patch applicator device. FIG. 12 is a perspective view of the actuator illustrated in FIGS. 11A and 11B. As shown, the actuator includes a bottom portion 128 and extensions 126. Generally, the actuator and outer body 110 are engaged to allow only substantially linear sliding motion between the two. As such the extensions 126 can engage the outer body via a groove, tube, glide path, or protrusion and cam path type connection. For example, the actuator 122 can have protrusions 124 that engage a linear cam path 111 of the body 110. It is contemplated that the configuration could be altered, e.g., placing the protrusion on the body and the path on the actuator 122. Further, although two extensions, protrusions, and cam paths are illustrated, three or more of each could be used.

As discussed previously, the piston 114 can have a number of configurations. Generally, the piston will be substantially cylindrical, as its round cross section will allow rotation within the body 110. FIG. 13 is a perspective view of a piston 114 of an exemplary patch applicator device. As shown, the piston 114 has protrusions 130, a top section 132, and a flange or widened region 138—all discussed above.

The piston 114 has a bottom surface 136 that is configured to push the patch downward. As shown, the surface 136 is convex, but it is contemplated that the surface can be flat or have other modifications such as a smooth or textured surface.

As discussed above in describing movement of the piston and actuator, the actuator pushes the piston upward to compress a spring or compressible member, but once the piston reaches a certain point in the cam path, the piston is released, thereby releasing energy stored in the now compressed spring and pushing the piston downward to apply the patch. It is contemplated that the piston and actuator can be engaged in different configurations to allow release of the piston from the actuator. In one embodiment, the piston can rotate within the outer body and with respect to the actuator.

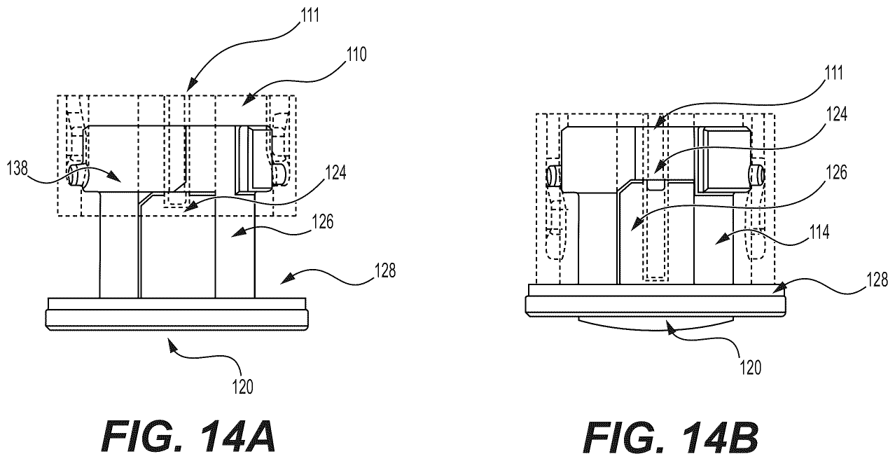

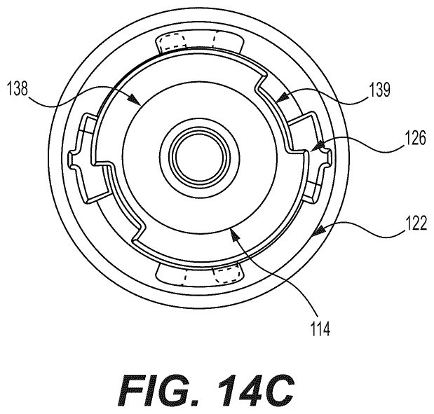

FIGS. 14A-14C illustrate interaction between an actuator 122 and piston 114 of the applicator, according to various embodiments. As shown, the actuator extensions 126 push on the flange or widened section 138 of the piston 114. However, the flange or widened section 138 does not extend around the entire periphery of the piston 114, but rather the widened section 138 has gaps 139 (FIG. 13B and FIG. 14C). As the protrusion 130 of the piston reaches the third downward directed section 143 of the cam path 140, the extensions 126 of the actuator reach the gaps 139 of the piston widened section 138. This effect is due to rotation of the piston 114 as the protrusion 130 moves through the looped cam path 140, along with linear movement of the actuator. As such, the piston 114 is released downward as the engagement between the piston protrusions 130 and cam path 140, and the engagement between the piston widened section 138 and actuator extensions 126, are simultaneously released.

It should be noted that although the components including the piston 114, actuator 122, and outer body are described with interacting protrusions 130 and 124 and cam paths 140 and 111, the location of the protrusions and cam paths may be altered. For example, the cam paths may be positioned on the actuator and/or piston with protrusions on an inner surface of the outer body 110, or combinations of such configurations.

Further, although the ring holder is attached to the bottom surface of the actuator, it is contemplated that the bottom surface of the actuator may be the skin contacting surface of the device, and the path may be secured to a top surface of the bottom portion 128 of the actuator (e.g., using the holder 60).

### Detailed Examples of Exemplary Patches

As discussed above, the applicator can be used to apply numerous types of medicament patches, but may be particularly desirable for application of microneedle devices. Accordingly, suitable patches including microneedles are described in more detail below. It is contemplated that the applicator and/or ring holder or sub-components may be provided as a kit or system including an applicator and patch, a patch and ring holder, or an applicator with one or multiple patches to be used with a reusable applicator. Suitable microneedles devices are further described in PCT Patent Application PCT/US2011/056856, titled “Silk fibroin-based microneedles and methods of making the same,” which was filed Oct. 19, 2011; PCT Patent Application PCT/US2019/025467, titled “Microneedle comprising silk fibroin applied to a dissolvable base,” which was filed Apr. 2, 2019; PCT Patent Application PCT/US2020/055139, titled “Silk Fibroin-Based Microneedles and Uses Thereof,” which was filed Oct. 9, 2020; PCT application PCT/US2021/033776, titled, “Compositions and devices for vaccine release and uses thereof,” which was filed May 21, 2021; PCT/US2022/030177, titled “Microneedle Vaccine Against Severe Acute Respiratory Syndrome Coronavirus 2 (SARS-CoV-2),” which was filed May 20, 2022, each of which is herein incorporated by reference in their entirety.

Suitable patches may preferably include silk fibroin-based microneedles and microneedle devices (e.g., microneedle arrays and patches) for the administration, transport, and release, e.g., controlled- or sustained-release, of a therapeutic agent, such as a vaccine, an antigen, and/or an immunogen (e.g., an influenza vaccine and/or a coronavirus vaccine, e.g., an mRNA-based vaccine) across a biological barrier, such as the skin, a mucous membrane, a buccal cavity, a tissue, or a cell membrane.

The term “administration” or “administering” includes routes of introducing a therapeutic agent to a subject to perform their intended function. In certain embodiments, the administration of the therapeutic agent, such as by a microneedle or microneedle device as described herein, may be repeated and the administrations may be separated by at least about 1 day, 2 days, 3 days, 5 days, 10 days, 15 days, 30 days, 45 days, 3 weeks, 4 weeks, 5 weeks, 6 weeks, 12 weeks, 2 months, 75 days, 3 months, or at least 6 months. In other embodiments, the administration of the therapeutic agent, such as by a microneedle or microneedle device as described herein, may be repeated annually. In other embodiments, the administration of the therapeutic agent, such as by a microneedle or microneedle device as described herein, may be repeated as often as necessary to achieve a therapeutic or prophylactic effect. Administration “in combination with” one or more further therapeutic agents includes simultaneous (concurrent) and consecutive administration in any order.

As used herein, a “subject” refers to a human or animal. Usually the animal is a vertebrate such as a primate, rodent, domestic animal, or game animal. Primates include chimpanzees, cynomolgus monkeys, spider monkeys, and macaques (e.g., Rhesus). Rodents include mice, rats, woodchucks, ferrets, rabbits and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish and salmon). In certain embodiments of the aspects described herein, the subject is a mammal (e.g., a primate, e.g., a human). A subject can be male or female. In certain embodiments, the subject is a mammal. The mammal can be a human, non-human primate, mouse, rat, dog, cat, horse, or cow, but are not limited to these examples. In addition, the methods and formulations described herein can be used to treat domesticated animals and/or pets. In some embodiments, the term “subject” is intended to include living organisms in which an immune response can be elicited (for example, mammals, for example, human).

In a particular embodiment, the subject is a human. A subject may be of any age. In an embodiment, the subject is an elderly human subject, e.g., 65 years of age or older. In an embodiment, a subject is a human subject who is not an elderly, e.g., less than 65 years of age. In an embodiment, a subject is a human pediatric subject, e.g., 18 years of age or less. In an embodiment, a subject is an adult subject, e.g., older than 18 years of age.

As used herein, the term “antigen” refers to refers to a molecule (e.g., a gene product (e.g., protein or peptide), pathogen fragment, whole pathogen, viral vector, or viral particle) capable of inducing a humoral immune response and/or cellular immune response, e.g., leading to the activation of B and/or T lymphocytes and/or innate immune cells and/or antigen presenting cells. Any macromolecule, including proteins or peptides, can be an antigen. Antigens can also be derived from genomic and/or recombinant DNA. For example, any DNA comprising a nucleotide sequence or a partial nucleotide sequence that encodes a protein capable of eliciting an immune response encodes an “antigen.” In some embodiments, an antigen does not need to be encoded solely by a full length nucleotide sequence of a gene, nor does an antigen need to be encoded by a gene at all. In some embodiments, an antigen can be synthesized or can be derived from a biological sample, e.g., a tissue sample, a tumor sample, a cell, or a fluid with other biological components. In some embodiments, an antigen can be derived from a virus, e.g., an inactivated virus, a viral like particle, or a viral vector. Antigens as used herein may also be mixtures of several individual antigens.

As used herein, the term “immunogen” refers to any substance (e.g., an antigen, combination of antigens, pathogen fragment, whole pathogen) capable of eliciting an immune response in an organism. An “immunogen” is capable of inducing an immunological response against itself after administration to a mammalian subject. The term “immunological” as used herein with respect to an immunological response, refers to the development of a humoral (antibody mediated) and/or a cellular (mediated by antigen-specific T cells or their secretion products) response directed against an immunogen in a recipient subject. Such a response can be an active response induced by administration of an immunogen or immunogenic peptide to a subject or a passive response induced by administration of antibody or primed T cells that are directed towards the immunogen. In some embodiments, an immunogen is a coronavirus antigen. In some embodiments, an immunogen is a coronavirus. In some embodiments, an immunogen is an influenza virus. In some embodiments, an immunogen is a viral vaccine (e.g., a monovalent (also called univalent) or a multivalent (also called polyvalent) vaccine, such as for coronavirus and/or influenza). In some embodiments, the vaccine (e.g., coronavirus vaccine and/or influenza vaccine) may be monovalent, bivalent, trivalent, quadrivalent (also called tetravalent), or pentavalent. In some embodiments, the immunogen is a replicating or non-replicating vaccine vector (e.g., comprises an adenovirus vector, an adeno-associated virus vector, an alpha virus vector, a herpesvirus vector, a measles virus vector, a poxvirus vector, or a vesicular stomatitis virus vector).

As used herein, the term “therapeutic agent” and “active agent” are art-recognized terms and refers to any chemical moiety that is a biologically, physiologically, or pharmacologically active substance that acts locally or systemically in a subject. Various forms of a therapeutic agent may be used which are capable of being released from the microneedles described herein into adjacent tissues or fluids upon administration to a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness, such as a viral infection; substances which affect the structure or function of the body; or pro-drugs, which become biologically active or more active after they have been placed in a physiological environment.

In certain embodiments, a therapeutic agent comprises, without limitation, a vaccine, an antigen, and/or an immunogen. In certain embodiments, a therapeutic agent comprises a coronavirus vaccine, antigen, and/or immunogen. In certain embodiments, a therapeutic agent comprises an influenza vaccine, antigen, and/or immunogen.

In certain embodiments, a therapeutic agent comprises, without limitation, an amino acid molecule, such as a peptide and/or a protein. In certain embodiments, a therapeutic agent comprises a recombinant protein vaccine.

In certain embodiments, a therapeutic agent comprises, without limitation, a nucleic acid molecule, such as a deoxyribonucleic acid (DNA) molecule and/or a ribonucleic acid (RNA) molecule. In particular embodiments, a therapeutic agent comprises an mRNA. In some embodiments, a therapeutic agent comprises a nucleic acid based vaccine, such as a DNA-based vaccine and/or a RNA-based vaccine. In some embodiments, a therapeutic agent comprises an mRNA-based vaccine.

As used herein, the term “vaccine” refers to any composition that will elicit a protective immune response in a subject that has been exposed to the composition. An immune response may include induction of antibodies and/or induction of a T-cell response. Usually, an “immune response” includes but is not limited to one or more of the following effects: the production or activation of antibodies, B cells, helper T cells, suppressor T cells, and/or cytotoxic T cells, directed specifically to an antigen or antigens included in or derived from the composition or vaccine of interest. Preferably, the subject will display either a therapeutic or a protective immunological (memory) response such that resistance to new infection will be enhanced and/or the clinical severity of the disease reduced. Such protection will be demonstrated by either a reduction in number or severity of, or lack of one or more of the clinical signs associated with the infection of the pathogen, in the delay of onset of viremia, in a reduced viral persistence, in a reduction of the overall viral load and/or in a reduction of viral excretion. In some embodiments, a “vaccine” refers to any preparation of an antigen or an immunogen (including subunit antigens, toxoid antigens, conjugate antigens, or other types of antigenic molecules, or nucleic acid molecules encoding the same) or a killed or live attenuated microorganism that, when introduced into a subject's body, affects the immune response to the specific antigen or microorganism by causing activation of the immune system against the specific antigen or microorganism (e.g., inducing antibody formation, T-cell responses, and/or B-cell responses). Generally, vaccines against microorganisms are directed toward at least part of a virus, bacteria, parasite, mycoplasma, or other infectious agent.

The term “therapeutically effective amount” refers to an amount of the composition as defined herein that is effective for preventing, ameliorating and/or treating a condition resulting from a disease as described herein, such as a viral infection.

The term “treatment” refers to therapeutic treatment as well as prophylactic or preventative measures to cure or halt or at least retard disease progress. Those in need of treatment include those already inflicted with a condition resulting from infection with a virus as described herein as well as those in which infection with a virus is to be prevented. Subjects partially or totally recovered form infection with a virus as described herein might also be in need of treatment. Prevention encompasses inhibiting or reducing the spread of a virus or inhibiting or reducing the onset, development or progression of one or more of the symptoms associated with infection with a virus described herein.

As used herein, the term “viruses” refers to an infectious agent composed of a nucleic acid encapsidated in a protein. Such infectious agents are incapable of autonomous replication (i.e., replication requires the use of the host cell's machinery). Viral genomes can be single-stranded (ss) or double-stranded (ds), RNA or DNA, and can or cannot use reverse transcriptase (RT). Additionally, ssRNA viruses can be either sense (+) or antisense (−). Exemplary viruses include, but are not limited to, dsDNA viruses (e.g., Adenoviruses, Herpesviruses, Poxviruses), ssDNA viruses (e.g., Parvoviruses), dsRNA viruses (e.g., Reo viruses), (+) ssRNA viruses (e.g., Picornaviruses, Toga viruses, Coronaviruses), (−) ssRNA viruses (e.g., Orthomyxoviruses, Rhabdoviruses), ssRNA-RT viruses, i.e., (+) sense RNA with DNA intermediate in life-cycle (e.g., Retroviruses), and dsDNA-RT viruses (e.g., Hepadnaviruses). In some embodiments, viruses can also include wild-type (natural) viruses, killed viruses, live attenuated viruses, modified viruses, recombinant viruses or any combinations thereof. Exemplary retroviruses include human immunodeficiency virus (HIV). Other examples of viruses include, but are not limited to, enveloped viruses, respiratory syncytial viruses, non-enveloped viruses (e.g., human papillomavirus (HPV)), bacteriophages, recombinant viruses, and viral vectors. The term “bacteriophages” as used herein refers to viruses that infect bacteria.

As used herein, the term “coronavirus” refers to a positive-sense ssRNA virus within the Coronaviridae family. A coronavirus may be an alphacoronavirus, a betacoronavirus, a gammacoronavirus, or a deltacoronavirus. A coronavirus can be a live wild-type virus, a live attenuated virus, an inactivated virus (e.g., a UV-inactivated virus), a chimeric virus, or a recombinant virus. Coronaviruses are known to infect humans and other animals (e.g., birds and mammals). Examples of coronaviruses include severe acute respiratory syndrome coronavirus (SARS-CoV), severe acute respiratory syndrome virus 2 (SARS-CoV-2), Middle East respiratory syndrome coronavirus (MERS-CoV), human coronavirus 229E (HCoV-229E), human coronavirus NL63 (HCoV-NL63), human coronavirus OC43 (HCoV-OC43), and human coronavirus HKU1 (HCoV-HKU1).

As used herein, the term “influenza virus” refers to a negative-sense ssRNA virus within the Orthomyxoviridae family. An influenza virus can be a live wild-type virus, a live attenuated virus, an inactivated virus, a chimeric virus, or a recombinant virus. Examples of influenza viruses include influenza A, influenza B, influenza C, and influenza D.

The microneedles described herein can be in any shape and/or geometry suitable for use in piercing a biological barrier, e.g., a layer of the skin, to enable release, e.g., controlled- or sustained-release, of a vaccine within a subject. Non-limiting examples of the shape and/or geometry of the microneedles include: a cylindrical shape, a wedge-shape, a cone-shape, a pyramid-shape, and/or an irregular-shape, or any combinations thereof.

As used herein, the term “release” and “controlled- or sustained-release” refers to the release of a vaccine, an antigen, and/or an immunogen (e.g., from a microneedle, microneedle device, formulation, composition, article, device, and preparation described herein, e.g., from a silk fibroin-based microneedle tip as described herein), such as a coronavirus vaccine, an influenza vaccine, or a combination thereof, over a period of time, e.g., for at least about 1 to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or 28 or more days, e.g., between about 4 days and about 25 days, between about 10 and about 20 days, between about 10 and about 15 days, between about 12 and about 16 days, e.g., between about 1-2 weeks, about 1-3 weeks, or about 1-4 weeks, e.g., between about 1 month to about 3 months). In some embodiments, the controlled- or sustained-release of a vaccine, such as a coronavirus vaccine and/or an influenza vaccine, over a time period of about 1 to about 14 days, e.g., about 1, 2, 3, 4, 5, 6, 7, 8, 9, 10, 11, 12, 13, or 14 days, by a microneedle, microneedle device, formulation, composition, article, device, or preparation as described herein can result, e.g., in broad-spectrum immunity in a subject. In some embodiments, the vaccine formulations and preparations comprising silk fibroin have controlled- or sustained-release properties (e.g., are formulated and/or configured to release a vaccine, e.g., into the skin of the subject, over a period of, or at least 1, 5, 10, 15, 30, 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; a period of, or at least, 1, 2, 3, 4, 5 years, or longer.

In some embodiments, a microneedle of the invention can comprise the following layers: (1) a backing material; (2) a dissolvable base; and (3) an implantable controlled- or sustained-release tip. For example, the microneedles described herein may include a backing material applied to a dissolvable base layer that supports a distal controlled- or sustained-release implantable tip comprising a silk fibroin and vaccine (e.g., an influenza vaccine and/or a coronavirus vaccine, e.g., an mRNA-based vaccine, antigen, and/or immunogen).

As used herein, the term “backing” refers to a material that is suitable for bonding to and/or adhering to a component of a microneedle. In some embodiments, a backing material is suitable for bonding to and/or adhering to the base (e.g., dissolvable base) of a microneedle described herein.

As used herein, the term “base” or “dissolvable base” refers to the layer that forms the base of the microneedles (e.g., functions as the support for the distal microneedle tips (e.g., silk fibroin tips) that are loaded with a vaccine, antigen, and/or immunogen (e.g., a coronavirus vaccine, an influenza vaccine, or a combination thereof)), and/or can also serve as a layer connecting adjacent microneedles to form a continuous microneedle array or microneedle patch. In some embodiments, at least 10%, 20%, 30%, 40%, 50%, 60%, 70%, 80%, 90% or more of the base is dissolved after application to a biological barrier, e.g., skin, mucous surface, or buccal cavity.

As used interchangeably herein, the terms “sustained-release tip,” “implantable sustained-release tip,” “implantable microneedle tip,” or “releasable tip” refers to the distal end, e.g., tip, of a microneedle capable of piercing a biological barrier, e.g., the skin, mucous surface, or buccal cavity, of a subject and being deposited within the biological barrier, a skin layer (e.g., the dermis). In embodiments, the tip comprises a silk fibroin protein in an amount sufficient to sustain the release of a vaccine, e.g., a coronavirus vaccine (e.g., a SARS-CoV-2 vaccine) and/or an influenza vaccine for a prolonged period of time, e.g., for at least about 1 day (e.g., about 1, 2, 3, 4, 5, 6, 7, 8, 9, 10, 11, 12, 13, or 14 or more days, e.g., between about 4 days and about 30 days, 5 days about 25 days, between about 10 days and about 20 days, between about 10 days and about 15 days, between about 4 days and about 14 days, between about 14 days and about 15 days, e.g., between about 1-2 weeks, about 1-3 weeks, or about 1-4 weeks, e.g., about 2-12 months). In some embodiments, the implantable sustained-release tip comprises a coronavirus vaccine, antigen, and/or immunogen. In some embodiments, the implantable sustained-release tip comprises an influenza vaccine, antigen, and/or immunogen.

As used herein, the term “microneedle” refers to a structure having at least two, more typically, three components, e.g., layers, for transport or delivery of a vaccine, an antigen, and/or an immunogen, across a biological barrier, such as the skin, tissue, or cell membrane. In some embodiments, a microneedle comprises a base (e.g., a dissolvable base as described herein), a tip (e.g., an implantable tip as described herein), and optionally, a backing material. In embodiments, a microneedle has dimension of between about 350 μm to about 1500 μm in height (e.g., between about 350 μm to about 1500 μm, e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm)). In some embodiments, the microneedle is fabricated to have any dimension and/or geometry to enable the deployment of a microneedle tip (e.g., a silk fibroin tip), e.g., an implantable sustained-release tip, at a depth between about 100 μm and about 900 μm (e.g., at a depth of about 800 μm) into the dermis layer of the skin for release, e.g., controlled- or sustained-release of a vaccine (e.g., a coronavirus vaccine and/or an influenza vaccine).

As used herein, the term “microneedle patch” and “microneedle array” refers to a device comprising a plurality of microneedles, e.g., silk fibroin-based microneedles, e.g., arranged in a random or predefined pattern, such as an array.

In some embodiments, the length of the microneedle can be between about 350 μm to about 1500 μm (e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 0 μm, about 1100 μm, about 1150 μm, about 1200 μm, about 1250 μm, about 1300 μm, about 1350 μm, about 1400 μm, about 1450 μm, about 1500 μm). In embodiments, the length of microneedles can be fabricated sufficiently long enough to enable delivery of an implantable tip comprising a vaccine, an antigen, and/or an immunogen for controlled- or sustained-release, as described herein, to the epidermis (e.g., about 10 μm to 120 μm below the skin surface), e.g., to induce an immune response. In some embodiments, the length of microneedles can be fabricated sufficiently long enough to enable delivery of an implantable tip comprising a vaccine, an antigen, and/or an immunogen for controlled- or sustained-release, as described herein, to the dermis (e.g., about 60 μm to about 2.1 mm below the skin surface). An skilled artisan can adjust the microneedle length for a number of factors, including, without limitations, tissue thickness, e.g., skin thickness, (e.g., as a function of age, gender, location on body, species (e.g., animal), drug delivery profile, diffusion properties of the vaccine, antigen, and/or immunogen for controlled- or sustained-release (e.g., the ionic charge and/or molecule weight, and/or shape of the vaccine, antigen, and/or immunogen for controlled- or sustained-release), or any combinations thereof. However, without wishing to be bound by theory, with an approximately 650 μm tall microneedle an implantable sustained-release tip may be deployed at a depth of between about 100 μm and about 600 μm within the dermis layer of the skin to a subject to achieve controlled- or sustained-release of vaccine from the tip. In some embodiments, the microneedle may be about 800 μm tall (e.g., between about 500 μm and 1200 μm tall).

Exemplary microneedles of the invention are depicted in FIGS. 5A-5B.

In some embodiments, a plurality of microneedles can be arranged in a random or predefined pattern to form a microneedle array and/or patch, as described herein. The patch may comprise a carrier, backing, or “handle” layer adhered to the back of the base (see, e.g., FIG. 4). This layer can provide structural support and an area by which the patch can be handled and manipulated without disturbing the needle array.

### Microneedle Array

The microneedle array may comprise about 121 needles in an 11×11 square grid with approximately 0.75 mm pitch. Individual needles are cones approximately 0.65 mm long with base diameter approximately 0.35 mm and included angle of approximately 30°. The tip of the needle must be sharp in order to penetrate the skin. The radius of curvature of the tip should ideally be no more than 0.01 mm.

### Backing

Exemplary backing materials that can be used in the fabrication of a microneedle of the invention include, but are not limited a solid support, e.g., a paper-based material, a plastic material, a polymeric material, or a polyester-based material (e.g., a Whatman 903 paper, a polymeric tape, a plastic tape, an adhesive-backed polyester tape, or other medical tape). In some embodiments, the backing comprises a Whatman 903 paper. In some embodiments, the backing comprises a polyester tape. In some embodiments, the polyester tape comprises an adhesive-backed polyester tape. In some embodiments, the backing material may be coated (e.g., at least on one side) with an adhesive suitable for bonding to and/or adhering to the dissolvable base of a microneedle described herein.

The backing materials used in the microneedles of the invention may have various properties, including, but not limited to, the ability to bond and/or adhere to the dissolving base layer to permit demolding. A backing material must be strong enough for the backing to maintain patch integrity, e.g., if the dissolving base layer has cracks or discontinuities. The backing material may be sufficiently flexible so as to conform, for example, to a non-flat surface, such as a skin surface. In particular, the backing must be flexible enough during wear time, such as after the patch is applied (e.g., pressed into) the skin. The backing may comprise and/or consist of a non-dissolving material, such that the backing maintains its integrity after patch application to a skin surface and during patch removal from a skin surface.

The backing may have any dimension suitable for application to a target skin surface. In some embodiments, the dimensions of the backing can be a 12 mm diameter circle. In some embodiments, the dimensions of the backing can be a 12 mm wide strip with a “handle” section of up to 12 mm length beyond the edge of the 12 mm×12 mm patch.

### Dissolvable Base

The dissolving base layer forms the base of the conical needles (e.g., functions as the support for the distal silk fibroin tips that are loaded with a vaccine, an antigen, and/or an immunogen). The dissolvable base layer can also function as a layer connecting adjacent needles to form a microneedle array or patch. In some embodiments, the dissolvable base layer comprises less than 98% (e.g., less than about 98%, less than about 90%, less than about 80%, less than about 70%, less than about 60%, less than about 50%, less than about less 40%, less than about 30%, less than about 20%, less than about 10%, less than about 9%, less than about 8%, less than about 7%, less than about 6%, less than about 5%, less than about 4%, less than about 3%, less than about 2%, less than about 1%) of the total amount (e.g., dose) of a vaccine, an antigen, and/or an immunogen comprises loaded into the microneedle and/or microneedle device. In some embodiments, the dissolvable base layer does not comprise, e.g., a detectable amount of, a vaccine, an antigen, and/or an immunogen. In some embodiments, dissolvable base layer is formulated to limit and/or reduce the amount of vaccine, antigen, and/or immunogen leakage (e.g., diffusion) from the silk fibroin tips into the dissolvable base layer, e.g., as compared to art known base layer formulations, e.g., base layer formulations comprising PAA. In some embodiments, a limit and/or reduce amount of vaccine, antigen, and/or immunogen leakage (e.g., diffusion) from the silk fibroin tips can be determined about 1 day, about 2 days, about 3 days, about 4 days, about 5 days, or about 6 days; about 1 week, about 2 weeks, or about 3 weeks; about 1 month, about 2 months, about 3 months, about 4 months, about 5 months, about 6 months, about 7 months, about 8 months, about 9 months, about 10 months, or about 11 months; or about 1 year or more after fabrication and storage (e.g., storage at about 4° C. (e.g., refrigeration), at about 25° C. (e.g., room temperature), at about 37° C. (e.g., body temperature), at about 45° C. and/or at about 50° C.), e.g., as compared to a base layer formulation comprising PAA.

The dissolvable base layer comprises a material that can dissolve into the skin, e.g., within the intended wear time (e.g., about five minutes). In some embodiments, the at least about 10%, 20%, 30%, 40%, 50%, 60%, 70%, 80%, 90%, or 100% of the dissolvable base layer is dissolved after application, e.g., to the skin, within the intended wear time (e.g., about 1 minute, about 2 minutes, about 3 minutes, about 4 minutes, about 5 minutes, about 6 minutes, about 7 minutes, about 8 minutes, about 9 minutes, or about 10 minutes or more).

The material used in the fabrication of the dissolvable base must be sufficiently strong enough to enable the microneedle to penetrate the skin, and be tough enough (e.g., not brittle) to also enable demolding. The dissolvable base material must be amenable to routine handling without catastrophic failure, and must retain its mechanical properties between demolding and application (e.g., not so hygroscopic that it melts due to ambient humidity). The dissolvable base layer material must be non-toxic and non-reactogenic at the doses used in a patch. In some embodiments, the dissolvable base layer comprises a water soluble component. In some embodiments, a dissolvable base layer, as described herein, has improved biocompatibility, e.g., as compared to a dissolvable base layer comprising poly (acrylic acid) (PAA). In some embodiments, the dissolvable base layer material causes a reduced inflammatory response and/or reduced tissue necrosis. In some embodiments, the dissolvable base layer material is not PAA, and induces a reduced inflammatory response and/or reduced tissue necrosis compared to PAA. In some embodiments, the dissolvable base layer material has a pH similar to that of the biological barrier into which it will be dissolved, e.g., a pH of about 4.0 to about 8.0

Non-limiting examples of materials that may be used to fabricate the dissolvable base layer include gelatin (e.g., hydrolyzed gelatin), polyethylene glycol (PEG), sucrose, low-viscosity carboxymethylcellulose (CMC), polyvinylpyrrolidone (PVP), polyvinyl alcohol (PVA), hyaluronate, maltose, and/or methyl cellulose. In some embodiments, the dissolvable base comprises one, two, three, four, five, six, seven, eight, or more (e.g., all) of gelatin, polyethylene glycol (PEG), sucrose, carboxymethylcellulose (CMC), polyvinylpyrrolidone (PVP), polyvinyl alcohol (PVA), hyaluronate, maltose, and methyl cellulose, e.g., at a concentration between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75%). In some embodiments, the dissolvable base does not comprise a therapeutic agent, as described herein.

In some embodiments, the dissolvable base comprises between about 10% and about 70% gelatin (e.g., hydrolyzed gelatin) (e.g., about 10%, about 20%, about 30%, about 40%, about 50%, about 60%, or about 70% gelatin).

In some embodiments, the dissolvable base comprises between about 1% and about 70% polyethylene glycol (PEG) (e.g., about 1%, about 2%, about 3%, about 4%, about 5%, about 6%, about 7%, about 8%, about 9%, about 10%, about 20%, about 30%, about 40%, about 50%, about 60%, or about 70% PEG).

In some embodiments, the dissolvable base comprises between about 1% and about 35% sucrose (e.g., about 1%, about 2%, about 3%, about 4%, about 5%, about 6%, about 7%, about 8%, about 9%, about 10%, about 15%, about 20%, about 25%, about 30%, or about 35% sucrose).

In some embodiments, the dissolvable base comprises between about 1% and about 35% CMC (e.g., about 1%, about 2%, about 3%, about 4%, about 5%, about 6%, about 7%, about 8%, about 9%, about 10%, about 15%, about 20%, about 25%, about 30%, or about 35% CMC).

In some embodiments, the dissolvable base comprises between about 10% and about 70% PVP (e.g., about 10%, about 20%, about 30%, about 40%, about 50%, about 60%, or about 70% PVP).

In some embodiments, the dissolvable base comprises between about 1% and about 35% PVA (e.g., e.g., about 1%, about 2%, about 3%, about 4%, about 5%, about 6%, about 7%, about 8%, about 9%, about 10%, about 15%, about 20%, about 25%, about 30%, or about 35% PVA).

In some embodiments, the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% hyaluronate).

In some embodiments, the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% maltose).

In some embodiments, the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% methyl cellulose).

In some embodiments, the dissolvable base layer may comprise 40% hydrolyzed gelatin, 10% Sucrose w/v in DI water. Optionally, the base layer may include 1% low-viscosity carboxymethylcellulose (CMC), which may reduce brittleness. In some embodiments, the dissolvable base layer may comprise polyvinylpyrrolidone (PVP) of 10 kD MW at up to 50% w/v in DI water; polyvinyl alcohol (PVA) 87% hydrolyzed at 13 kD MW at up to 20% in DI water; or CMC at up to 10% in DI water. The following combinations may also be suitable for use in the fabrication of a dissolvable base layer: 30% PVP and 10% PVA; 37% PVP, 5% PVA, and 15% sucrose; or various other proportions of PVP, PVA, and sucrose.

In some embodiments, the dissolvable base layer is approximately 12 mm square and 0.75 mm thick. In some embodiments, the dissolvable base layer can cover the entire patch. In some embodiments, the dimension of the base layer can be a 12 mm diameter circle, or a 12×12 mm square.

### Implantable Sustained-Release Tip

In embodiments, the implantable sustained-release tip can be fabricated from silk fibroin and may comprise a vaccine, an antigen, and/or an immunogen as described herein (e.g., an influenza vaccine and/or a coronavirus vaccine, e.g., an mRNA-based vaccine). In some embodiments, the implantable sustained-release tip can be designed to be deployed into the dermis layer of the skin (e.g., not into the subcutaneous space), as the population of professional antigen presenting cells in the dermis is much higher than in the subcutaneous space. In humans, the dermis ranges from about 1000-2000 μm (e.g., about 1-2 mm) thick based on location and patient age and health. In rodents, the dermis is much thinner (e.g., mice ˜100-300 μm, and rats ˜800-1200 μm). Without wishing to be bound by theory, with a 650 μm tall microneedle an implantable sustained-release tip may be deployed at a depth of between about 100 μm and about 600 μm to achieve the controlled- or sustained-release of a vaccine, an antigen, and/or an immunogen as described herein (e.g., an influenza vaccine and/or a coronavirus vaccine, e.g., an mRNA-based vaccine).

Without being bound by theory, the molecular weight of the silk fibroin solution used in the fabrication of a microneedle described herein can function as a control factor to modulate the controlled- or sustained-release of a vaccine, an antigen, and/or an immunogen (e.g., an influenza vaccine and/or a coronavirus vaccine, e.g., an mRNA-based vaccine) from the tip. In some embodiments, a higher molecular weight silk fibroin solutions can favor a slower controlled- or sustained-release (e.g., reducing the amount of an initial burst (e.g., the amount released on Day 0) by at least about 10% and then releasing additional antigen over at least about the next 4 days.). In some embodiments, the controlled- or sustained-release of a vaccine, an antigen, and/or an immunogen (e.g., an influenza vaccine and/or a coronavirus vaccine, e.g., an mRNA-based vaccine) from the tip may be over at least about 4 days (e.g., about 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks). In some embodiments, controlled- or sustained-release occurs over about 1 week to about 2 weeks.

In embodiments, the silk fibroin solution used in the fabrication of a microneedle described herein can be a low molecular weight silk fibroin composition comprising a population of silk fibroin fragments having a range of molecular weights, characterized in that: no more than 15% of the total number of silk fibroin fragments in the population has a molecular weight exceeding 200 kDa, and at least 50% of the total number of the silk fibroin fragments in the population has a molecular weight within a specified range, wherein the specified range is between about 3.5 kDa and about 120 kDa, or between about 5 kDa and about 125 kDa. Stated another way, the silk fibroin solution used in the fabrication of a microneedle described herein can comprise a population of silk fibroin fragments having a range of molecular weights, characterized in that: no more than 15% of the total moles of silk fibroin fragments in the population has a molecular weight exceeding 200 kDa, and at least 50% of the total moles of the silk fibroin fragments in the population has a molecular weight within a specified range, wherein the specified range is between about 3.5 kDa and about 120 kDa, or between about 5 kDa and about 125 kDa. (see, e.g., WO2014/145002, incorporated herein by reference herein).

Exemplary silk fibroin (e.g., regenerated silk fibroin) solutions may have different molecular weight profiles are shown as determined by size exclusion chromatography (SEC) methods (see, e.g., FIG. 5). In some embodiments, the silk fibroin solutions can be prepared, e.g., according to established methods. In some embodiments, pieces of cocoons from the silkworm Bombyx mori were first boiled in 0.02 M Na2CO3 to remove sericin protein which is present in unprocessed, natural silk, prior to analysis by SEC. In some embodiments, silk fibroin composition can be a composition or mixture produced by degumming cocoons from the silkworm Bombyx mori at an atmospheric boiling temperature for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter. In one embodiment, the silk fibroin composition can be a composition or mixture produced by degumming silk cocoon at an atmospheric boiling temperature in an aqueous sodium carbonate solution for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter.

In some embodiments, the silk fibroin solution may be a 10-minute boil (10 MB), a 60-minute boil (60 MB), a 120-minute boil (120 MB), a 180-minute boil (180 MB), or a 480-minute boil (480 MB) silk fibroin solution (see, e.g., FIG. 5). In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 10 MB silk fibroin solution. In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 60 MB silk fibroin solution. In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 120 MB silk fibroin solution.

In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 180 MB silk fibroin solution. In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 480 MB silk fibroin solution.

Without being bound by theory, the primary tunability of the implantable sustained-release tip is its crystallinity, measured via beta-sheet content (intermolecular and intramolecular β-sheet). This impacts the solubility of the silk tip matrix and the ability of antigen to be retained. With the increased β-sheet content, the tip also becomes more mechanically strong. Specific vaccine release profiles are achieved through modulation of the crystallinity and the diffusivity of the silk matrix. This is accomplished through both silk input material and formulation as well as post-treatment to increase crystallinity (e.g. water annealing, methanol/solvent annealing). In some embodiments, the implantable controlled- or sustained-release microneedle tip comprises a beta-sheet content of between about 10% and about 60% (e.g., about 10%, about 20%, about 30%, about 40%, about 50%, about 60%), e.g., as based on a “crystallinity index,” e.g., a “crystallinity index” known in the art. In some embodiments, the implantable controlled- or sustained-release microneedle tip can be formulated as a particle (e.g., a microparticle and/or a nanoparticle).

### Dimensions of the Implantable Sustained-Release Tip

The methods provided herein can be used to fabricate silk fibroin-based implantable sustained-release tips of any dimensions, e.g., ranging from about 75 μm to about 800 μm in height/length (e.g., about 75, about 100 μm, about 125 μm, about 150 μm, about 250 μm to about 300 μm, about 300 μm to about 350 μm, about 350 μm to about 400 μm, about 400 μm to about 450 μm, about 450 μm to about 500 μm, about 500 μm to about 550 μm, about 550 μm to about 600 μm, about 600 μm to about 650 μm, about 650 μm to about 700 μm, about 700 μm to about 750 μm, about 750 μm, to about 800 μm), and/or having a tip radius of about 10 μm or less (e.g., between about 1 μm and about 10 μm, e.g., about 1 μm or less, about 2 μm or less, about 3 μm or less, about 4 μm or less, about 5 μm or less, about 6 μm or less, about 7 μm or less, about 8 μm or less, about 9 μm or less, or about 10 μm or less). In some embodiments, the implantable tip can have a diameter of any size, e.g., based upon the type of biological barrier (e.g., skin layer) intended to be pierced by the tip. In embodiments, the tip can have a dimension (e.g., a diameter) ranging from about 50 nm to about 50 μm (e.g., about 50 nm to about 250 nm, about 250 nm to about 500 nm, about 500 to about 750 nm, about 750 nm to about 1 μm, about 1 μm to about 5 μm, about 5 μm to about 10 μm, about 10 μm to about 15 μm, about 15 μm to about 20 μm, about 20 μm to about 25 μm, about 25 μm to about 30 μm, about 30 μm to about 35 μm, about 35 μm to about 40 μm, about 40 μm to about 45 μm, or about 45 μm to about 50 μm). It can be understood that there is no fundamental limitation preventing the sustained-release tips from having even smaller diameters (e.g., the limit of silk replica casting has been demonstrated with a resolution of tens of nm, see, e.g., Perry et al., 20 Adv. Mat. 3070 (2008)).

In some embodiments, the sharpness of the implantable sustained-release tip point is described herein in terms of tip radius. The molds used in the fabrication of the microneedles described herein are designed to have a tip radius between about 0.5 μm to about 10 μm (e.g., about 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, or 10 μm). In some embodiments, the tip radius is between about 20 μm to about 25 μm (e.g., about 20 μm, 21 μm, 22 μm, 23 μm, 24 μm, or 25 μm). Without being bound by theory, it can be understood that blunter needles may require more force to penetrate the epidermis. In embodiments, other dimensions of the implantable sustained-release tip may be controlled by the shape of the mold and fill volume. In some embodiments, the implantable sustained-release tip have an included angle between about 5 degrees and about 45 degrees (e.g., about 5, 6, 7, 8, 9, 10, 15, 20, 25, 30, 35, 40, or 45 degrees). In some embodiments, the implantable sustained-release tip can have an included angle between about 15 degrees and 45 degrees (e.g., about 15 degrees, about 16 degrees, about 17 degrees, about 18 degrees, about 19 degrees, about 20 degrees, about 21 degrees, about 22 degrees, about 23 degrees, about 24 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 36 degrees, about 37 degrees, about 38 degrees, about 39 degrees, about 40 degrees, about 41 degrees, about 42 degrees, about 43 degrees, about 44 degrees, or about 45 degrees.

In embodiments, the height of the implantable sustained-release tip may depend on the formulation and print volume, which can influence the surface tension and drying kinetics. In some embodiments, the height of the implantable sustained-release tip may extend to half of the full height of the microneedle. In some embodiments, the height of the implantable sustained-release tip is between about 75 μm to about 475 μm (e.g., about 75, about 100 μm, about 125 μm, about 150 μm, about 175 μm, about 200 μm, about 225 μm, about 250 μm, about 275 μm, about 300 μm, about 325 μm, about 375 μm, about 400 μm, about 425 μm, or about 475 μm). In some embodiments, the base of the tip comprises a thin “shell”-like layer roughly between about 5-10 μm thick (e.g., about 5, 6, 7, 8, 9, or 10 μm thick). In some embodiments, the implantable sustained-release tip may dry to a more solid construct with minimal “shell” wherein the height may be closer to 150 μm (e.g., between about 50 μm and about 200 μm) and the thickness >50 μm (e.g., between about 25 μm and about 75 μm).

Further, the microneedles of the present invention can take advantage of art known techniques developed, e.g., to functionalize silk fibroin (e.g., active agents such as dyes and sensors). See, e.g., U.S. Pat. No. 6,287,340, Bioengineered anterior cruciate ligament; WO 2004/000915, Silk Biomaterials & Methods of Use Thereof; WO 2004/001103, Silk Biomaterials & Methods of Use Thereof; WO 2004/062697, Silk Fibroin Materials & Use Thereof; WO 2005/000483, Method for Forming inorganic Coatings; WO 2005/012606, Concentrated Aqueous Silk Fibroin Solution & Use Thereof; WO 20111005381, Vortex-Induced Silk fibroin Gelation for Encapsulation & Delivery; WO 20051123114, Silk-Based Drug Delivery System; WO 2006/076711, Fibrous Protein Fusions & Uses Thereof in the Formation of Advanced Organic/Inorganic Composite Materials; U.S. Application Pub. No. 2007/0212730, Covalently immobilized protein gradients in three-dimensional porous scaffolds; WO 2006/042287, Method for Producing Biomaterial Scaffolds; WO 2007/016524, Method for Stepwise Deposition of Silk Fibroin Coatings; WO 2008/085904, Biodegradable Electronic Devices; WO 20081118133, Silk Microspheres for Encapsulation & Controlled Release; WO 20081108838, Microfluidic Devices & Methods for Fabricating Same; WO 20081127404, Nanopattemed Biopolymer Device & Method of Manufacturing Same; WO 20081118211, Biopolymer Photonic Crystals & Method of Manufacturing Same; WO 20081127402, Biopolymer Sensor & Method of Manufacturing Same; WO 20081127403, Biopolymer Optofluidic Device & Method of Manufacturing the Same; WO 20081127401, Biopolymer Optical Wave Guide & Method of Manufacturing Same; WO 20081140562, Biopolymer Sensor & Method of Manufacturing Same; WO 20081127405, Microfluidic Device with Cylindrical Microchannel & Method for Fabricating Same; WO 20081106485, Tissue-Engineered Silk Organs; WO 20081140562, Electroactive Bioploymer Optical & Electro-Optical Devices & Method of Manufacturing Same; WO 20081150861, Method for Silk Fibroin Gelation Using Sonication; WO 20071103442, Biocompatible Scaffolds & Adipose-Derived Stem Cells; WO 20091155397, Edible Holographic Silk Products; WO 20091100280, 3-Dimensional Silk Hydroxyapatite Compositions; WO 2009/061823, Fabrication of Silk Fibroin Photonic Structures by Nanocontact Imprinting; WO 20091126689, System & Method for Making Biomaterial Structures.

In various embodiments, the silk fibroin-based microneedle tips can further comprise at least one additional therapeutic agent, wherein the additional therapeutic can be dispersed throughout the microneedle or form at least a portion of the microneedle tip. In some embodiments, the additional therapeutic agent is useful in the treatment of a viral infection described herein. Optionally the silk fibroin-based microneedle tips can further comprise an excipient and/or adjuvant, as described herein.

### Viruses, Antigens, and Immunogens

The present invention provides, in some embodiments, the delivery, e.g., the controlled- or sustained-delivery, of various therapeutic agents, such as vaccines, antigens, and/or immunogens derived from a virus that is a member of the family Orthomyxovirus, e.g., by a formulation, composition, articles, device, preparations, microneedle and/or microneedle device (e.g., a microneedle patch) described herein and/or according to a method described herein. In some embodiments, a vaccine, a microneedle, and/or a microneedle device (e.g., a microneedle patch) described herein may comprise a negative-sense ssRNA virus and/or an RNA virus, such as an influenza virus. In some embodiments, the vaccine, antigen, and/or immunogen comprises a nucleic acid (e.g., a DNA and/or RNA) derived from an influenza virus. In some embodiments, the vaccine, antigen, and/or immunogen comprises an amino acid (e.g., a peptide and/or protein) derived from an influenza virus. In some embodiments, the influenza vaccine, antigen, and/or immunogen comprise an inactivated and/or a live attenuated virion, or split virion, of an influenza virus. In some embodiments, the vaccine and/or the microneedle comprises a non-replicating viral antigen.

In particular, the invention contemplates a vaccine, a microneedle, and/or a microneedle device (e.g., a microneedle patch) comprising an influenza virus vaccine, antigen, and/or immunogen. The influenza virus is a RNA virus (e.g., a linear negative-sense single stranded RNA virus). There are four known genera of influenza virus, each containing a single type (e.g., Influenza A, B, C, and D). Influenza viruses can continuously change and are subject to both antigenic drift and antigenic shift. Exemplary influenza strains are further described in the Examples (see, e.g., Tables 1 and 2).

Influenza A can be divided into subtypes on the basis of two proteins on the surface of the virus: hemagglutinin (HA) and neuraminidase (NA). Influenza A comprises 18 known HA subtypes, referred to herein as H1-H18, and 11 known NA subtypes, referred to herein as N1-N11. Many different combinations of HA and NA proteins may be found on the surface of the influenza A virus. For example, an “H1N1 virus” designates an influenza A virus subtype comprising an H1 protein and an N1 protein. Exemplary influenza A virus subtypes confirmed to infect humans include, but are not limited to, H1N1, H3N2, H2N2, H5N1, H7N7, H1N2, H9N2, H7N2, H7N3, H10N7, and H7N9. The H1N1 virus and H3N2 virus are currently in general circulation among humans.

Exemplary Influenza B viruses may belong to, e.g., the B/Yamagata lineage and/or the B/Victoria lineage.

### Vaccines

Non-limiting examples of influenza vaccines for use in the microneedles and microneedle devices (e.g., microneedle patches) described herein can include a commercial vaccine, such as a seasonal vaccine, a pandemic vaccine, and/or a universal vaccine; egg-based vaccines, cell-culture based vaccines; recombinant vaccines; live attenuated, inactivated whole virus, split virion, and/or protein subunit vaccines; and adjuvanted vaccines. Various commercial influenza vaccines are listed below. Additionally, influenza vaccines comprising an mRNA, a DNA, a viral vector, and/or a virus-like particle (VLP) are suitable for use in the microneedles and microneedle devices (e.g., microneedle patches) described herein. In some embodiments, the influenza vaccine may target matrix protein 1, matrix protein 2 (M2e), and/or nucleoprotein (NP) of an influenza virus.

TABLE 1
Exemplary Vaccines

| Vaccine | Manufacturer |
|---|---|
| Seasonal Influenza Vaccines | |
| Fluzone High Dose | Sanofi Pasteur |
| Fluzone Quadrivalent | Sanofi Pasteur |
| Fluzone Intradermal Quadrivalent | Sanofi Pasteur |
| Afluria/Fluvax | Seqirus |
| Agriflu | Seqirus |
| Fluad | Seqirus |
| Flucelvax | Seqirus |
| Fluvirin | Seqirus |
| Aggripal | Seqirus |
| FluMist Quadrivalent | MedImmune |
| Flublok | Protein Sciences (Sanofi Pasteur) |
| FluLaval | GlaxoSmithKline |
| Fluarix | GlaxoSmithKline |
| Influvac | Mylan |
| Preflucel | Nanotherapeutics |
| Anflu | Sinovac Biotech |
| Pandemic Influenza Vaccines | |
| Influenza Virus Vaccine, H5N1 | Sanofi Pasteur |
| Pandemrix | GlaxoSmithKline |
| Panflu | Sinovac Biotech |
| Panflu 1 | Sinovac Biotech |

### Vaccine Formulations and Composition for Controlled- or Sustained-Release

At least one vaccine, antigen, and/or immunogen described herein (e.g., at least one vaccine, antigen, and/or immunogen derived from an influenza virus described herein) can be incorporated into a variety of formulations, compositions, articles, devices, and/or preparations for administration, e.g., to achieve controlled- and/or sustained release. More particularly, at least one vaccine, antigen, and/or immunogen described herein (e.g., at least one vaccine, antigen, and/or immunogen derived from an influenza virus described herein) can be formulated into formulations, compositions, articles, devices, and/or preparations by combination with appropriate, pharmaceutically acceptable carriers or diluents, and can be formulated into preparations in semi-solid, solid, or liquid formats. In some embodiments, the formulations, compositions, articles, devices, and/or preparations described herein comprise silk fibroin. Exemplary formulations, compositions, articles, devices, and/or preparations comprise: a microneedle (e.g., a microneedle device, e.g., a microneedle patch, e.g., as described herein), an implantable device (e.g., a pump, e.g., a subcutaneous pump), an injectable formulation, a depot, a gel (e.g., a hydrogel), an implant, and a particle (e.g., a microparticle and/or a nanoparticle). As such, administration of the compositions can be achieved in various ways, including intradermal, intramuscular, transdermal, subcutaneous, or intravenous administration. Moreover, the formulations, compositions, articles, devices, and/or preparations can be formulated and/or administered to achieve controlled- and/or sustained release of the at least one vaccine, antigen, and/or immunogen described herein (e.g., at least one vaccine, antigen, and/or immunogen derived from an influenza virus described herein).

In some embodiments, the vaccine (e.g., the influenza vaccine) is administered, e.g., substantially sustained, over a period of, or at least 1, 5, 10, 15, 30, 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; a period of, or at least, 1, 2, 3, 4, 5 years, or longer. In one embodiment, the vaccine (e.g., the influenza vaccine) is administered as a controlled- or sustained release formulation, dosage form, or device. In certain embodiments, the vaccine (e.g., the influenza vaccine) is formulated for continuous delivery, e.g., intradermal, intramuscular, and/or intravenous continuous delivery. In some embodiments, the composition or device for the controlled- or sustained-release of the vaccine is chosen from: a microneedle (e.g., a microneedle device, e.g., a microneedle patch), an implantable device (e.g., a pump, e.g., a subcutaneous pump), an injectable formulation, a depot, a gel (e.g., a hydrogel), an implant, or a particle (e.g., a microparticle and/or a nanoparticle). In one embodiment, the vaccine (e.g., the influenza vaccine) is in a silk-based controlled- or extended release dosage form or formulation (e.g., a microneedle described herein). In one embodiment, the vaccine (e.g., the influenza vaccine) is administered via an implantable device, e.g., a pump (e.g., a subcutaneous pump), an implant, an implantable tip of a microneedle, or a depot. The delivery method can be optimized such that a vaccine (e.g., an influenza vaccine and/or a coronavirus vaccine, e.g., an mRNA-based vaccine) dose as described herein (e.g., a standard dose) is administered and/or maintained in the subject for a pre-determined period (e.g., a period of, or at least: 1, 5, 10, 15, 30, 45 minutes; 1, 2, 3, 4, 5, 10, 24 hours 1, 2, 3, 4, 5, 6, 7, 8, 9, 10, 11, 12, 13, 14 days; 1, 2, 3, 4, 5, 6, 7, 8 weeks; 1, 2, 3, 4, 5, 6, 7, 8, 9, 10, 11 months; 1, 2, 3, 4, 5 years, or longer). The substantially sustained or extended release of the vaccine (e.g., the influenza vaccine) can be used for prevention or treatment of a viral infection (e.g., an influenza viral infection) for a period of hours, days, weeks, months, or years.

The present invention provides, in some embodiments, formulations, compositions, articles, devices, and/or preparations of the invention can be formulated and/or configured for controlled- or sustained-release of a at least one vaccine, antigen, and/or immunogen (e.g., at least one vaccine, antigen, and/or immunogen derived from an influenza virus described herein) in an amount (e.g., a dosage) and/or over a time period sufficient to result in an immune response (e.g., a cellular immune response and/or a humoral immune response) to the virus, e.g., the influenza virus, in the subject.

In some embodiments, the formulations, compositions, articles, devices, and/or preparations of the invention can be formulated and/or configured for controlled- or sustained-release of a at least one vaccine, antigen, and/or immunogen (e.g., at least one vaccine, antigen, and/or immunogen derived from an influenza virus described herein) in an amount (e.g., a dosage) and/or over a time period sufficient to result in broad spectrum immunity in the subject.

The substantially continuously or extended release delivery or formulation of the vaccine (e.g., the influenza vaccine) can be used for prevention or treatment of a viral infection (e.g., an influenza viral infection) for a period of hours, days, weeks, months, or years.

In some embodiments, at least one vaccine, antigen, and/or immunogen described herein can be added to the silk fibroin solution, e.g., before forming the silk fibroin microneedles or microneedle devices described herein. In embodiments, a silk fibroin solution can be mixed with a vaccine, antigen, and/or immunogen, and then used in the fabrication of an implantable microneedle tip, e.g., by the process of filling and/or casting, drying, and/or annealing to produce a microneedle having any of the desired material properties, as described herein.

Without being bound by theory, the ratio of silk fibroin to vaccine, antigen, and/or immunogen in an implantable tip of a microneedle influences their release. In some embodiments, increased silk concentration in the implantable tip favors a slower release and/or greater antigen retention within the tip. Any concentration of silk may be used, as long as the concentration allows for printing and has the mechanical strength sufficient to pierce the skin.

In some embodiments, silk fibroin can be used at a concentration ranging from about 1% w/v to about 10% w/v (e.g., about 1, 2, 3, 4, 5, 6, 7, 8, 9, or 10% w/v) in the fabrication of a microneedle, or a component thereof, as described herein.

### Exemplary Excipients

In addition, the formulations, compositions, articles, devices, and/or preparations can be formulated with common excipients, diluents or carriers for administered by the intradermal, intramuscular, transdermal, subcutaneous, or intravenous routes. In some embodiments, the formulations, compositions, articles, devices, and/or preparations can be administered, e.g., transdermally, and can be formulated as controlled- or sustained-release dosage forms and the like. The formulations, compositions, articles, devices, and/or preparations described herein can be administered alone, in combination with each other, or they can be used in combination with other known therapeutic agents.

Suitable formulations for use in the present invention are found in Remington's Pharmaceutical Sciences (1985). Moreover, for a review of methods for drug delivery, see, Langer (1990) Science 249:1527-1533. The formulations, compositions, articles, devices, and/or preparations described herein can be manufactured in a manner that is known to those of skill in the art, e.g., by mixing, dissolving, granulating, dragee-making, levigating, emulsifying, encapsulating, entrapping or lyophilizing processes. The following methods and excipients are merely exemplary and are in no way limiting.

The silk fibroin formulations used in the fabrication of the microneedles described herein may include excipients. In embodiments, inclusion of an excipient may be for the purposes of improving the stability of an incorporated vaccine, antigen, and/or immunogen; to increase silk matrix porosity and diffusivity of the vaccine, antigen, and/or immunogen from the formulation, composition, article, device, preparation, and/or microneedle, e.g., microneedle tip; and/or to increase crystallinity/beta-sheet content of silk matrix to render the silk-material insoluble.

Exemplary excipients include, but are not limited to, a sugar or a sugar alcohol (e.g., sucrose, trehalose, sorbitol, mannitol, or a combination thereof), a divalent cation (e.g., Ca2+, Mg2+, Mn2+, and Cu2+), and/or buffers. In some embodiments, the concentration of an excipient can be used to modify the porosity of the matrix, e.g., with sucrose being used as the most common excipient for this purpose. Excipients may also be added to favor silk self-assembly into order beta-sheet secondary structure, and such excipients generally can participate in hydrogen bonding or charge interactions with silk to achieve this effect. Non-limiting examples of excipients that can be used to favor silk self-assembly into order beta-sheet secondary structure include monosodium glutamate (e.g., L-glutamic acid), lysine, sugar alcohols (e.g., sorbitol and/or glycerol), and solvents (e.g., DMSO, methanol, and/or ethanol).

In some embodiments, the sugar or the sugar alcohol is sucrose present in an amount less than 70% (w/v), less than 60% (w/v), less than 50% (w/v), less than 40% (w/v), less than 30% (w/v), less than 20% (w/v), less than 10% (w/v), less than 9% (w/v), less than 8% (w/v), less than 7% (w/v), less than 6% (w/v), or 5% (w/v) or less, e.g., immediately before drying.

In some embodiments, the sugar or the sugar alcohol is sucrose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is trehalose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is sorbitol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is glycerol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the vaccine preparation further comprising a divalent cation. In some embodiments, the divalent cation is selected from the group consisting of Ca2+, Mg2+, Mn2+, and Cu2+. In some embodiments, the divalent cation is present in the preparation, e.g., immediately before drying, in an amount between 0.1 mM and 100 mM. In some embodiments, the divalent cation is present in the preparation, e.g., immediately before drying, in an amount between 10-7 and 10-4 moles per standard dose of viral immunogen. In some embodiments, the divalent cation is present in the preparation immediately before drying in an amount between 10-10 to 2×10-3 moles.

In some embodiments, the vaccine preparation further comprises poly (lactic-co-glycolic acid) (PGLA).

In some embodiments, the viral vaccine preparation further comprising a buffer, e.g., immediately before drying. In some embodiments, the buffer has buffering capacity between pH 3 and pH 8, between pH 4 and pH 7.5, or between pH 5 and pH 7. In some embodiments, the buffer is selected from the group consisting of HEPES and a CP buffer. In some embodiments, the buffer is present in the preparation, e.g., immediately before drying, in an amount between 0.1 mM and 100 mM. In some embodiments, the buffer is present in an amount between 10-7 and 10-4 moles per standard dose of viral immunogen. In some embodiments, the buffer is present in an amount between 10-10 to 2×10-3 moles.

In addition, the vaccine can also be formulated as a depot, gel, or hydrogel preparation. Such long acting formulations can be administered by implantation (for example subcutaneously or intramuscularly) or by intramuscular injection. Thus, for example, the vaccine can be formulated with suitable polymeric or hydrophobic materials (for example as an emulsion in an acceptable oil) or ion exchange resins, or as sparingly soluble derivatives, for example, as a sparingly soluble salt.

In one embodiment, the vaccine is administered via an implantable infusion device, e.g., a pump (e.g., a subcutaneous pump), an implant or a depot. Implantable infusion devices typically include a housing containing a liquid reservoir which can be filled transcutaneously by a hypodermic needle penetrating a fill port septum. The medication reservoir is generally coupled via an internal flow path to a device outlet port for delivering the liquid through a catheter to a patient body site. Typical infusion devices also include a controller and a fluid transfer mechanism, such as a pump or a valve, for moving the liquid from the reservoir through the internal flow path to the device's outlet port.

In some embodiments, the vaccine can be packages and/or formulated as a particle, e.g., a microparticle and/or a nanoparticle. Typically nanoparticles are from 10, 15, 20, 25, 30, 35, 45, 50, 75, 100, 150 or 200 nm or 200-1,000, e.g., 10, 15, 20, 25, 30, 35, 45, 50, 75, 100, 150, or 200, or 20 or 30 or 50-400 nm in diameter. Smaller particles tend to be cleared more rapidly form the system. Therapeutic agents, including vaccines, can be entrapped within or coupled, e.g., covalent coupled, or otherwise adhered, to nanoparticles.

Lipid- or oil-based nanoparticles, such as liposomes and solid lipid nanoparticles and can be used to can be used to deliver therapeutic agents, e.g., vaccines, described herein. Solid lipid nanoparticles for the delivery of therapeutic agents are descripbed in Serpe et al. (2004) Eur. J. Pharm. Bioparm. 58:673-680 and Lu et al. (20060 Eur. J. Pharm. Sci. 28: 86-95. Polymer-based nanoparticles, e.g., PLGA-based nanoparticles can be used to deliver agents described herein. These tend to rely on biodegradable backbone with the therapeutic agent intercalated (with or without covalent linkage to the polymer) in a matrix of polymer. PLGA is a widely used in polymeric nanoparticles, see Hu et al. (2009) J. Control. Release 134:55-61; Cheng et al. (2007) Biomaterials 28:869-876, and Chan et al. (2009) Biomaterials 30:1627-1634. PEGylated PLGA-based nanoparticles can also be used to deliver therapeutic agents, see, e.g., Danhhier et al., (2009) J. Control. Release 133:11-17, Gryparis et al (2007) Eur. J. Pharm. Biopharm. 67:1-8. Metal-based, e.g., gold-based nanoparticles can also be used to deliver therapeutic agents. Protein-based, e.g., albumin-based nanoparticles can be used to deliver agents described herein. In some embodiments, a therapeutic agent can be bound to nanoparticles of human albumin.

A broad range of nanoparticles are known in the art. Exemplary approaches include those described in WO2010/005726, WO2010/005723 WO2010/005721, WO2010/121949, WO2010/0075072, WO2010/068866, WO2010/005740, WO2006/014626, 7,820,788, 7,780,984, the contents of which are incorporated herein in reference by their entirety.

### Dosages

Any dosage amount (e.g., a standard dose and/or a fractional dose) of a vaccine, antigen, and/or immunogen that is capable of eliciting an immune response (e.g., immunogenicity and/or broad-spectrum immunity) in a subject, e.g., when administered by a microneedle of the invention, may be used according to the methods described herein. In some embodiments, dose, e.g., the standard dose (e.g., human dose) for a vaccine, an antigen, and/or an immunogen (e.g., an influenza vaccine and/or a coronavirus vaccine, e.g., an mRNA-based vaccine) is between about 0.1 μg and about 65 μg (e.g., between about 0.1 μg and about 10 μg, between about 0.1 μg and about 1 μg, between about 0.5 μg and about 5 μg, between about 5 μg and about 10 μg, between about 10 μg and about 20 μg, between about 20 μg and about 30 μg, between about 30 μg and about 40 μg, about 40 μg and about 50 μg, about 50 μg and about 65 μg, e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg). In some embodiments, the dose, e.g., standard human dose, for a vaccine described herein (e.g., an influenza vaccine and/or a coronavirus vaccine, e.g., an mRNA-based vaccine) is approximately between about 1 μg and about 30 μg per strain, e.g., between about 5 μg and about 30 μg per strain (e.g., about 5, 6, 7, 8, 9, 10, 11, 12, 13, 14, 15, 16, 17, 18, 19, 20, 21, 22, 23, 24, 25, 26, 27, 28, 29, or 30 μg per strain). In some embodiments, the dose, e.g., fractional dose, for a vaccine described herein (e.g., an influenza vaccine and/or a coronavirus vaccine, e.g., an mRNA-based vaccine) is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose). It is known in the art, that there is clinical precedent for dose-sparing when delivering influenza vaccine to the intradermal space (e.g., Fluzone ID), and this this dose is about 9 μg per strain. Accordingly, in some embodiments the total dosage amount of an influenza vaccine (e.g., Fluzone ID) that can be delivered by a microneedle of the invention can be between about 5 μg and 13 μg (e.g., about 5 μg, about 6 μg, about 7 μg, about 8 μg, about 9 μg, about 10 μg, about 11 μg, about 12 μg, or about 13 μg).

Without wishing to be bound by theory, the total dosage amount (e.g., a standard dose) of a vaccine, antigen, and/or immunogen to be administered by a microneedle described herein can be divided between a plurality of microneedles (e.g., within a patch), such that a microneedle tip can comprises less than about 1% of the total dosage amount (e.g., in an array comprising about 121 microneedles), or at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of the total dosage amount. In some embodiments, an implantable microneedle tip, as described herein, can comprise about 0.1 μg to about 65 μg (e.g., about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1 μg to about 10 μg, about 10 μg to about 20 μg, about 20 μg to about 30 μg, about 30 μg to about 40 μg, about 40 μg to about 50 μg, about 50 μg to about 65 μg) of a vaccine, antigen, and/or immunogen, as described herein.

In some embodiments, the vaccine dosage amount loaded into a microneedle patch can be manipulated via the concentration of antigen in the formulated solution that forms the needle tips, the volume of solution dispensed into each needle tip, and the total number of needles (the former two are more convenient means of varying dose). The dosage released into the skin is related to deployment efficiency (the portion of needle tips that are left behind in the skin after the patch is removed), and also the release profile over time and the residence time of the tips within the skin. Because of the continuous sloughing of skin from the epidermis, deeper deployment within the skin is related to longer residence time. Therefore, it is desirable to maximize the penetration depth of the needle tip (up to a limit defined by the depth of pain receptors within the skin, e.g., at a depth of between about 100 μm and about 600 μm), and also to have the antigen spatially concentrated toward the tip of the needle.

The formulations, compositions, articles, devices, and/or preparations described herein, including the implantable sustained-release tip formulation, are designed to not only sustain release of vaccine antigen over the duration, e.g., of tip retention in the dermis, but to also maintain stability of antigen during this period of time (e.g., at least about 1-2 weeks). In some embodiments, approximately 95-100% of the total dosage amount incorporated, e.g., in a formulation, composition, article, device, preparation, and/or microneedle described herein, can be expected to be available for delivery, e.g., into a subject, e.g., into a tissue of a subject, such as the skin, a mucous membrane, an organ tissue, a buccal cavity, a tissue, or a cell membrane. Without being bound by theory, successful deployment of a microneedle into the skin is at least about 50% and can be as high as 100% of an array (e.g., upon application at least about 50%, 60%, 70%, 80%, 90% or more (e.g., 100%) of the total number of microneedle comprising an array are successfully deployed within, e.g., the skin, for controlled- or sustained-release of a vaccine antigen). In some embodiments, a portion of antigen may not be released from the silk tips during the duration of deployment.

### Uses

The invention also provides methods for delivering a vaccine, an antigen, and/or an immunogen (e.g., an influenza vaccine and/or a coronavirus vaccine, e.g., an mRNA-based vaccine) across a biological barrier (e.g., the skin). Such methods can include providing a formulation, composition, article, device, preparation, and/or microneedle described herein. For example, such methods can include providing at least one microneedle or at least one microneedle device described herein, wherein the microneedle or the microneedle device comprises a silk fibroin-based implantable tip having at least one vaccine, antigen, and/or an immunogen (e.g., an influenza vaccine and/or a coronavirus vaccine, e.g., an mRNA-based vaccine); causing the microneedle or microneedle device to penetrate into the biological barrier (e.g., the skin); and allowing the vaccine, antigen, and/or an immunogen to be released from the implantable tips over a period of at least about 4 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks). In some embodiments, the vaccine, antigen, and/or an immunogen is released into the biological barrier through the degradation and/or dissolution of the implantable microneedle tips. In some embodiments, the microneedle or microneedle device is configured to administer the vaccine, antigen, and/or an immunogen in an amount and/or a duration that results in broad-spectrum immunity in the subject, e.g., an immunity against one or more viral antigens not present in the implantable sustained-release tip, e.g., an immunity against a drifted strain not present in the implantable sustained-release tip.

The invention also provides a method for providing broad-spectrum immunity to a virus, e.g., an influenza virus, in a subject, said method comprising administering a vaccine (e.g., an influenza vaccine and/or a coronavirus vaccine, e.g., an mRNA-based vaccine) in an amount (e.g., a dosage) and/or over a time period sufficient to result in broad-spectrum immunity to a virus, e.g., results in an immune response (e.g., a cellular immune response and/or a humoral immune response) to a drifted strain of the virus, in the subject. In some embodiments, the vaccine is administered in a composition for the controlled- or sustained-release of the vaccine (e.g., for the controlled- or sustained-release of one or more viral antigens as described herein). In some embodiments, the vaccine is administered by a device for the controlled- or sustained-release of the vaccine (e.g., for the controlled- or sustained-release of one or more viral antigens as described herein). The vaccine can be administered into a subject, e.g., in to a tissue or cavity of the subject chosen from skin, mucosa, organ tissue, muscle tissue or buccal cavity.

In some embodiments, the methods described herein comprise administering a in an amount (e.g., a dosage) and/or over a time period sufficient to result in one or more of: (i) exposure in the subject to one or more antigens in the vaccine in an amount and/or period of time to result in broad spectrum immunity, e.g., to result in an immune response (e.g., a cellular immune response and/or a humoral immune response) to a drifted strain of the virus, in the subject; or (ii) a level of one or more antigens in the subject that is substantially steady, e.g., about 20%, 15%, 10%, 5%, or 1% to an amount, e.g., minimum amount, needed to result in an immune response (e.g., a cellular immune response and/or a humoral immune response) to the one or more antigens. In some embodiments, the composition or device for the controlled- or sustained-release of the vaccine is chosen from: a microneedle (e.g., a microneedle device, e.g., a microneedle patch, e.g., as described herein), an implantable device (e.g., a pump, e.g., a subcutaneous pump), an injectable formulation, a depot, a gel (e.g., a hydrogel), an implant, or a particle (e.g., a microparticle and/or a nanoparticle).

In some embodiments, the vaccine is administered, e.g., released by the composition or device for the controlled- or sustained-release of the vaccine, e.g., into the subject, in order to maintain a vaccine dosage (e.g., an antigen concentration) for a period of time sufficient to result in broad spectrum immunity, e.g., to result in an immune response (e.g., a cellular immune response and/or a humoral immune response) to a drifted strain of the virus, in the subject (e.g., wherein the period of time is about 1 to 21 days, e.g., about 5 to 10 days or about 5 to 7 days, e.g., about 1, 2, 3, 4, 5, 6, 7, 8, 9, 10, 11, 12, 13, 14, 15, 16, 17, 18, 19, 20, or 21 days). The composition or device for the controlled- or sustained-release of the vaccine can maintain antigen release and/or level in the subject over a sustained period of time. In some embodiments the composition or device for the controlled- or sustained-release of the vaccine maintains a continuous or non-continuous antigen release into the subject over a sustained period of time. The vaccine can administered, e.g., released by the composition or device for the controlled- or sustained-release, over a period of time comprising at least about one week, e.g., about 1-2 weeks, about 1-3 weeks, or about 1-4 weeks. In some embodiments, the vaccine is administered, e.g., released by the composition or device for the controlled- or sustained-release, over a period of time comprising at least about 4 days (e.g., about 4, 5, 6, 7, 8, 9, 10, 11, 12, 13, 14, 15, 16, 17, 18, 19, or 20 days, or more, e.g., between about 4 days and about 2 weeks, between about 4 days and about 1 week).

The vaccine can be administered in a dosage comprising between about 0.1 μg and about 65 μg per strain, e.g., 0.2 μg and about 50 μg per strain (e.g., about each of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg per strain). In some embodiments, at least about 1% of the dosage of the vaccine (e.g., at least about 0.5% to about 10%, at least about 5% to about 15% at least about 10% to about 20% of the dosage), e.g., released by the composition or device for the controlled- or sustained-release of the vaccine, e.g., into the subject, is maintained over a period of time comprising at least about 4 days (e.g., about 4, 5, 6, 7, 8, 9, 10, 11, 12, 13, or 14 days, or more, e.g., between about 4 days and about 2 weeks, between about 4 days and about 1 week).

In some embodiments, the vaccine is administered, e.g., released by the composition or device for the controlled- or sustained-release, in a plurality of fractional doses of a total dose (e.g., a standard dose) over a time period, e.g., such that an immune response and/or broad-spectrum immunity is achieved, wherein the amount of the vaccine administered in each of the fractional doses is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the vaccine.

In some embodiments, the vaccine is administered, e.g., released by the composition or device for the controlled- or sustained-release of the vaccine, e.g., into the skin of the subject, in a plurality of doses equivalent to a percentage of a total dose (e.g., a percentage of a standard dose) over a time period, e.g., such that broad-spectrum immunity is achieved, wherein the amount of the vaccine administered in each of the plurality of doses is about X %, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 125, 150, 175, 200, 300, 400, or 500 or more, of the total dose (e.g., a standard dose) of the vaccine.

The vaccine can be administered according to any of the methods described herein such that broad-spectrum immunity is achieved, e.g., such that an immune response, e.g., a cellular immune and/or humoral immune response to a drifted strain is achieved.

Without wishing to be bound by theory, a subject exposed to and/or infected with a first influenza virus can develop an immune response (e.g., a cellular immune and/or humoral immune response) resulting in the creation of an antibody against that first influenza virus. As antigenic changes (e.g., mutations) accumulate in the first influenza virus over time, the subject's antibodies created against the first influenza virus may no longer recognize the drifted virus (e.g., the antigenically different strain). Using the methods, dosage regimens, microneedles, and microneedle devices described herein, broad-spectrum immunity can be conferred to a subject exposed to, infected with, and/or at risk of infection with an influenza virus. Further, using the methods, dosage regimens, microneedles, and microneedle devices described herein, improved immunogenicity and/or broad-spectrum immunity can be conferred to a subject, e.g., as compared to traditional burst release administration of vaccine. For example, improved immunogenicity and/or broad-spectrum immunity detectable in a subject can be greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to traditional burst release administration of vaccine, e.g., the administration of a single-dose or a bolus administration of the vaccine.

In some embodiments, the implantable sustained-release tip or the vaccine comprises a first influenza strain and administration of a dose of the first influenza strain (e.g., a first influenza A, B, C, and/or D strain as described herein) to the subject results in the development of broad-spectrum immunity to a second influenza strain (e.g., a drifted influenza A, B, C, and/or D strain as described herein) not present in the implantable sustained-release tip or the vaccine.

In some embodiments, the subject (e.g., the human subject) is a pediatric subject, an adult subject, or an elderly subject. The subject may have been exposed to, infected with, and/or at risk of infection with an influenza virus (e.g., a particular strain of an influenza virus). Such a risk may be due to the health status or age of the subject and/or travel to a region where a particular strain of influenza virus is prevalent.

In some embodiments, the invention provides methods of providing a controlled- or sustained-release of a vaccine in a subject. The controlled- or sustained-release of the vaccine can achieve an improved immunogenicity and/or broad-spectrum immunity, as compared to traditional burst release administration of vaccine. Without wishing to be bound by theory, an method of administering a vaccine described herein and/or a controlled- or sustained-release rate, e.g., by a composition and/or a microneedle described herein, that mimics the natural exposure pattern of a subject (e.g., a human subject) to a virus can provide enhanced immunity and/or broad-spectrum immunity to a subject, as compared to traditional single-dose vaccine administration modalities.

In some embodiments, a desired amount of at least one vaccine, antigen, and/or immunogen (e.g., an influenza vaccine and/or a coronavirus vaccine, e.g., an mRNA-based vaccine) can be released from the microneedle (e.g., implantable microneedle tip) described herein in a sustained manner over a pre-defined period of time. In some embodiments, at least about 5% of a vaccine, an antigen, and/or an immunogen (e.g., an influenza vaccine and/or a coronavirus vaccine, e.g., an mRNA-based vaccine), e.g., at least about 10%, at least about 15%, at least about 20%, at least about 30%, at least about 40%, at least about 50%, at least about 60%, at least about 70%, at least about 80%, at least about 90%, at least about 95%, or at least about 97%, about 98%, or about 99%, or 100% of the vaccine, antigen, and/or an immunogen (e.g., an influenza vaccine and/or a coronavirus vaccine, e.g., an mRNA-based vaccine), can be released from the microneedle (e.g., implantable microneedle tips) over a pre-defined period of time. In such embodiments, the desired amount (e.g., a dose, such as a standard dose of a vaccine) of the vaccine, antigen, and/or immunogen (e.g., an influenza vaccine and/or a coronavirus vaccine, e.g., an mRNA-based vaccine) can be released from the microneedle over seconds, minutes, hours, months and/or years. In some embodiments, the desired amount (e.g., a dose, such as a standard dose of a vaccine) of the vaccine, antigen, and/or immunogen (e.g., an influenza vaccine and/or a coronavirus vaccine, e.g., an mRNA-based vaccine) can be released from the microneedle upon insertion into a biological barrier, e.g., within 5 seconds, within 10 seconds, within 30 seconds, within 1 minute, within 2 minutes, within 3 minutes, within 4 minutes, within 5 minutes or longer. In some embodiments, the desired amount (e.g., a dose, such as a standard dose of a vaccine) of the vaccine, antigen, and/or immunogen (e.g., an influenza vaccine and/or a coronavirus vaccine, e.g., an mRNA-based vaccine) can be released from the microneedle over a period of at least about 1 hour, at least about 2 hours, at least about 3 hours, at least about 6 hours, at least about 12 hours, at least about 1 day, at least about 2 days, at least about 3 days, at least about 4 days, at least about 5 days, at least about 6 days, at least about 1 week, at least about 2 weeks, at least about 1 month, at least about 2 months, at least about 3 months, at least about 6 months or longer. In some embodiments, the desired amount (e.g., a dose, such as a standard dose of a vaccine) of the vaccine, antigen, and/or immunogen (e.g., an influenza vaccine and/or a coronavirus vaccine, e.g., an mRNA-based vaccine) can be released from the microneedle over about 1 year or longer.

In some embodiments, the invention provides methods for enhancing an immune response to a virus in a subject. In some embodiments, the presence of a cell-mediated immunological response can be determined by any art-recognized methods, e.g., proliferation assays (CD4+ T cells), CTL (cytotoxic T lymphocyte) assays (see Burke, supra; Tigges, supra), or immunohistochemistry with tissue section of a subject to determine the presence of activated cells such as monocytes and macrophages after the administration of an immunogen. One of skill in the art can readily determine the presence of humoral-mediated immunological response in a subject by any well-established methods. For example, the level of antibodies produced in a biological sample such as blood can be measured by western blot, ELISA or other methods known for antibody detection. In some embodiments, an elevated hemagglutination inhibition (HAI) antibody titer is detectable in the blood of the subject for the duration of a complete flu season post immunization.

In some embodiments, the immune response and/or the broad-spectrum immunity is a cellular immune and/or humoral immune response comprising: (i) an elevated hemagglutination inhibition (HAI) antibody titer detectable in the blood of the subject, e.g., detectable at least 3, 4, 5, 6, 7, 8, 9, 10, 11, 12, 13, 14, 15, 16, 17, 18, 19, 20, 21, 22, 23, 24, 25, 26, 27, 28, 29, and/or 30-weeks or more post immunization; (ii) an elevated anti-influenza IgG titer detectable in the blood of the subject, e.g., detectable at least 1, 2, 3, 4, 5, 6, 7, 8, 9, 10, 11 and/or 12-months or more post immunization; and/or (iii) a level of antibody secreting plasma cells (ASC) against the virus, e.g., the influenza virus, detectable in the bone marrow of the subject, e.g., detectable at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, and/or 34-weeks or more post immunization. In some embodiments, the elevated HAI antibody titer is to a drifted influenza A, B, C, and/or D strain. In some embodiments, the elevated anti-influenza IgG titer is to a drifted influenza A, B, C, and/or D strain. In some embodiments, the immune response is a cellular immune response comprising an increase in the level of IFNγ secreting cell in the blood of the subject, e.g., at least 2, 3, 4, 5, 6, 7, 8, 9, 10, 11, and/or 12-weeks or more post immunization, e.g., by a microneedle described herein.

In some embodiments, the elevated HAI antibody titer, the elevated anti-influenza IgG titer, the level of antibody secreting plasma cells (ASC) against the virus, and/or the level of IFNγ secreting cells detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.

In some embodiments, broad-spectrum immunity can be characterized by measuring the percent seroconversion in a subject. For example, broad-spectrum immunity can comprise a percent seroconversion, e.g., based on the elevated HAI antibody titer detectable in the blood of the subject, e.g., at 6-month post immunization greater than about 20% (e.g., 20%, 25%, 30%, 35%, 40%, 45%, 50%, 55%, 60%, 65%, 70%, 75%, 80%, 85%, 90%, or 95% or more, e.g., 100%). Such a level of seroconversion associated with broad-spectrum immunity conferred by using the methods, dosage regimens, microneedles, and microneedle devices described herein can be greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to a level of seroconversion obtained by traditional burst release administration of vaccine, e.g., the administration of a single-dose or a bolus administration of the vaccine.

### Combination Therapies

The microneedles and microneedle devices (e.g., microneedle patches) described herein may be manufactured by precision filling of each individual microneedle tip to enable different patterns of vaccine delivery, dosing schemes, and combination administration of a vaccine with an additional therapeutic agent. The methods of immunization, vaccine delivery, and dosing described herein may comprise combination administration of a vaccine with an additional therapeutic agent. In some embodiments, an additional therapeutic agent may be formulated in the same tip as a vaccine. In some embodiments, an additional therapeutic agent may be formulated with the vaccine. For example, adjuvants to boost immune response to co-delivered antigen could be delivered in the same microneedle tip and/or vaccine. Without wishing to be bound by theory, such a combination therapy could include adjuvants to drive stronger cellular immune responses and/or mucosal responses. Moreover, additional influenza antigens could be delivered for heterologous “prime/boost-like” immunization, e.g., primary immunization with an HA antigen from various influenza strains and a boost (e.g., provided via controlled- or sustained-release or distinct kinetic pattern from “prime”) with a different antigen (e.g., a drifted strain, a hemagglutinin stem, m2e protein, or NA).

Formulation compatibility may limit whether two given therapeutic agents can be co-formulated to be dispensed into the same needle tip. In case co-formulation is not possible, the manufacturing process can be adapted in order to dispense a first formulation into a portion of the needle array and then dispense a second formulation into a different portion of the needle array. Different formulations can also receive different process treatments after filling. For instance, if the first formulation will be for controlled- or sustained-release and the silk will be rendered less soluble via water annealing, while the second formulation will be for burst release with no annealing, the second formulation can be dispensed after the annealing step. The manufacturing approach is flexible so other process sequences are possible.

In some embodiments, the invention also provides methods for combination therapies, wherein a microneedle or microneedle device of the invention can be fabricated to administer at least one additional therapeutic agent. Various forms of a therapeutic agent can be used which are capable of being released from the microneedles described herein into adjacent tissues or fluids upon administration to a subject. In some embodiments, an additional therapeutic agent can be included within the base layer and/or within the implantable tip.

Examples of additional therapeutic agents that can be used according to the methods of the invention, e.g., incorporated into a microneedle of the invention, e.g., during fabrication, include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., mRNA sequences or antisense oligonucleotides that bind to a target nucleic acid sequence), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

### Exemplary Kits

In certain embodiments, the invention relates to a package or kit comprising a microneedle described herein (e.g., a microneedle including a vaccine, antigen, and/or an immunogen as described herein, such as an influenza virus). In some embodiments, the invention relates to a package or kit comprising a vaccine described herein (e.g., a vaccine, antigen, and/or an immunogen as described herein, such as an influenza virus). In some embodiments, the kit can further comprise an additional therapeutic for combination therapy with the microneedle. In some embodiments, the kits can further comprise a disinfectant (e.g., an alcohol swab). In some embodiments, such packages, and kits described herein can be used for vaccination purposes, e.g., to achieve broad-spectrum immunity in a subject as described herein.

What is claimed is:

1. A medicament patch and applicator system, comprising:
a medicament patch comprising a flexible sheet of material;
an applicator for holding and applying the medicament patch to a patient's skin, comprising:
an outer body portion having a substantially cylindrical shape and a hollow interior;
a piston portion slidably connected with the hollow interior of the outer body portion; and
a compressible member positioned within the outer body and configured to apply downward pressure to the piston;
wherein the medicament patch is held in a ring holder at a bottom portion of the applicator and is pushed downward through the ring holder and onto an object held in contact with a bottom surface of the ring holder.

2. The system of claim 1, wherein the compressible member comprises a spring.

3. The system of claim 1, wherein the ring holder is removable from the applicator.

4. The system of claim 1, wherein the ring holder is integrally connected to the applicator.

5. The system of claim 1, wherein the piston and hollow interior of the outer body portion are slidably connected with at least one protrusion and at least one cam path.

6. The system of claim 1, further comprising:
an actuator positioned below the piston and being slidably engaged with the hollow interior and extending from a bottom portion of the outer body such that upward pressure on the actuator pushes the piston upward and compresses the compressible member; and
wherein the piston and hollow interior of the outer body portion are slidably connected with a protrusion and a cam path, and wherein the cam path forms a continuous loop such that upward pressure on the piston portion causes the piston to compress the compressible member moving the piston upward into the hollow interior of the outer body portion until the protrusion reaches a top portion of the cam path and engages a downward directed portion of the cam path, thereby releasing the piston into a downward portion of the continuous loop to release the piston and force the piston downward.

7. The system of claim 6, wherein the protrusion extends from a side wall, and the cam path is formed along an interior wall of the outer body portion.

8. The system of claim 6, wherein the actuator is slidably engaged with the hollow interior along a connection between at least one protrusion of the actuator and at least one cam path of the hollow interior.

9. The system of claim 6, wherein the piston is rotatably mobile within the hollow interior.

10. The system of claim 1, wherein the piston has a curved bottom surface to impact the medicament patch.

11. The system of claim 1, wherein the ring holder has a solid rim portion and an open center portion.

12. The system of claim 11, wherein the solid rim portion is substantially circular.

13. The system of claim 11, wherein the medicament patch and ring holder are sized such that the medicament patch is larger than the open center portion in at least one dimension such that the medicament patch can sit on top of the ring holder without passing through the center portion.

14. The system of claim 11, wherein the medicament patch has a degree of flexibility such that downward pressure on the medicament patch caused by releasing of the piston causes the medicament patch to bend sufficiently to allow the medicament patch to pass through the center portion of the ring holder.

15. The system of claim 1, wherein the medicament patch is a flat sheet in the form of a circular, triangular, square, polygonal, or oval shape.

16. The system of claim 1, wherein the medicament patch has one or more notches along its periphery to facilitate bending of the medicament patch near the periphery of the medicament patch.

17. The system of claim 1, wherein the medicament patch is a microneedle patch.

18. A medicament patch and applicator system, comprising:
a medicament patch comprising a flexible sheet of material;
an applicator for holding and applying the medicament patch to a patient's skin, comprising:
an outer body portion and a hollow interior;
a piston portion slidably connected with the hollow interior of the outer body portion; and
a ring holder replacably connected to a bottom portion of the applicator, the ring holder including a solid circular rim and an open center portion,
wherein the medicament patch is held in the ring holder at the bottom portion of the applicator and is pushed downward through the open center portion of the ring holder and onto an object held in contact with a bottom surface of the ring holder.

19. The system of claim 18, wherein the medicament patch has a degree of flexibility such that downward pressure on the medicament patch caused by releasing of the piston causes the medicament patch to bend sufficiently to allow the medicament patch to pass through the center portion of the ring holder.

20. The system of claim 18, wherein the piston has a curved bottom surface to impact the medicament patch.

21. The system of claim 18, wherein the medicament patch has one or more notches along its periphery to facilitate bending of the medicament patch near the periphery of the medicament patch.

22. A medicament patch and applicator system, comprising:
a medicament patch comprising a flexible sheet of material;
an applicator for holding and applying the medicament patch to a patient's skin, comprising:
an outer body portion having a substantially cylindrical shape and a hollow interior;
a mobile piston portion connected with the hollow interior of the outer body portion; and
a ring holder detachably connectable to a bottom portion of the applicator,
wherein the medicament patch is held in the ring holder at the bottom portion of the applicator and is pushed downward through an open center portion of the ring holder and onto an object held in contact with a bottom surface of the ring holder.

23. The system of claim 22, wherein the medicament patch has a degree of flexibility such that downward pressure on the medicament patch caused by releasing of the piston causes the medicament patch to bend sufficiently to allow the medicament patch to pass through the center portion of the ring holder.

24. The system of claim 22, wherein the piston has a curved bottom surface to impact the medicament patch.

25. The system of claim 22, wherein the medicament patch has one or more notches along its periphery to facilitate bending of the medicament patch near the periphery of the medicament patch.

26. A medicament patch and holder assembly, comprising:
a medicament patch comprising a flexible sheet of material and a group of microneedles on one surface of the medicament patch; and
a ring holder including a solid circular rim and an open center portion, wherein the medicament patch and ring holder are sized such that the medicament patch is held over the open center portion.

27. The assembly of claim 26, wherein the medicament patch is a flat sheet in the form of a circular, triangular, square, polygonal, or oval shape.

28. The assembly of claim 26, wherein the medicament patch has a degree of flexibility such that downward pressure on the medicament patch causes the medicament patch to bend sufficiently to allow the medicament patch to pass through the center portion of the ring holder

29. The assembly of claim 26, wherein the medicament patch has one or more notches along its periphery to facilitate bending of the medicament patch near the periphery of the medicament patch.
